(19) United States
(12) Patent Application Publication
(10) Pub. No.: US 2021/0346329 A1
(43) Pub. Date: Nov. 11, 2021

(54) TCA CYCLE INTERMEDIATES AND METHODS OF USE THEREOF

(71) Applicant: IMBRIA PHARMACEUTICALS, INC., Boston, MA (US)

(72) Inventors: Andrew D. Levin, Newton, MA (US); David-Alexandre Gros, Rancho Santa Fe, CA (US); Jaikrishna Patel, Cary, NC (US)

(21) Appl. No.: 17/282,581

(22) PCT Filed: Oct. 10, 2019
(86) PCT No.: PCT/US2019/055643

§ 371 Date: Apr. 2, 2021

Related U.S. Application Data
(60) Provisional application No. 62/744,251, filed on Oct. 11, 2018.
(60) Provisional application No. 62/771,289, filed on Nov. 26, 2018.
(60) Provisional application No. 62/799,064, filed on Jan. 31, 2019.
(60) Provisional application No. 62/900,921, filed on Sep. 16, 2019.

(51) Int. Cl. A61K 31/194; A61K 47/18; A61K 47/02; A61K 9/00

(52) U.S. Cl. CPC A61K 31/194; A61K 47/183; A61K 9/0019; A61K 9/0053; A61K 47/02

(57) ABSTRACT

The invention generally relates to non-oral formulations of TCA cycle intermediates and methods of using such formulations for treating metabolic disorders. In certain embodiments, the invention provides therapeutic compositions containing succinate formulated for non-oral administration and methods of using such compositions to treat metabolic disorders. The invention also provides combination therapies that include compositions containing succinate formulated for non-oral administration and compositions containing citrate (optionally formulated for oral administration) and methods of using such combination therapies to treat metabolic disorders.

### CROSS-REFERENCE TO RELATED APPLICATIONS

This application claims the benefit of, and priority to, U.S. Provisional Application No. 62/744,251, filed Oct. 11, 2018; U.S. Provisional Application No. 62/771,289, filed Nov. 26, 2018; U.S. Provisional Application No. 62/799,064, filed Jan. 31, 2019; and U.S. Provisional Application No. 62/900,921, filed Sep. 16, 2019, the contents of each of which are incorporated herein by reference.

### FIELD OF THE INVENTION

The invention generally relates to TCA cycle intermediates and methods of using such compositions for treating metabolic disorders.

### BACKGROUND

Inherited metabolic disorders, such as propionic acidemia (PA) and methylmalonic acidemia (MMA), cause a variety of life-threatening symptoms, including seizures, strokes, and damage to the brain, heart, and liver. PA and MMA result from enzymatic deficiencies that impair the body's ability to convert certain fats and amino acids into succinate, an intermediate in the tricarboxylic acid (TCA) cycle. Consequently, individuals with PA or MMA must maintain a low-protein diet for their entire lives, and in some patients, even strict adherence to the diet fails to prevent neurological damage and other symptoms.

Efforts to treat afflicted individuals by providing additional succinate have failed. Unadulterated succinate is poorly bioavailable, so providing succinate in quantities sufficient to remedy metabolic disorders is difficult. Thus, despite our understanding of the molecular basis of metabolic disorders like PA and MMA, these genetic disorders remain highly debilitating and often fatal.

### SUMMARY

The invention overcomes the challenge of delivering succinate at doses therapeutically effective to treat metabolic disorders such as PA and MMA by providing combination therapies that include multiple intermediates of the TCA cycle. By providing two or more TCA cycle intermediates to a subject, compositions and methods of the invention rely on the body's natural metabolism pathways to restore levels of succinate and allow delivery of a larger bolus of succinate than is possible with a single agent. In addition, because the compositions can provide various TCA cycle intermediates, they are useful in treating a broad spectrum of metabolic diseases, disorders, and conditions. For example, combinations of succinate and citrate are useful for treating PA and MMA, but other combinations may be used to treat different conditions.

The use of multiple TCA cycle intermediates allows flexibility in the formulation and administration of the compositions of the invention. Each TCA cycle intermediate may independently be provided to the subject by the optimal route of administration for the particular form of the intermediate. Thus, succinate-containing combinations may include non-oral delivery of succinate salts or oral delivery of succinate derivatives that have improved bioavailability. Moreover, in succinate-citrate combinations, both intermediates may be given orally, both may be given non-orally, or one may be given orally and the other may be given non-orally.

The invention also provides succinate-containing compositions formulated for non-oral administration. The compositions, which include formulations for subcutaneous or intravenous administration, have superior bioavailability to oral formulations. The non-oral formulations of succinate may be used in the combination therapies of the invention.

Compositions containing succinate or other TCA cycle intermediates can be readily administered to infants, including newborns. Many metabolic disorders such as PA and MMA are due to genetic defects, so the consequences of errant metabolism become manifest at birth when the child no longer has access to a nutritional supply from the mother. Therefore, early intervention is necessary to rectify metabolic imbalances before they lead to permanent symptoms. Because the invention provides succinate-containing compositions formulated for non-oral administration, such compositions do not require active suckling from the baby to deliver therapeutically effective doses of succinate. Consequently, treatment of infants can begin immediately to help prevent development of disease symptoms.

In an aspect, the invention provides compositions containing one or more TCA cycle intermediates or prodrugs, analogs, or derivatives thereof formulated for non-oral administration. The TCA cycle intermediate may be citrate, isocitrate, alpha-ketoglutarate, succinyl-coenzyme A, succinate, fumarate, malate, or oxaloacetate. Preferably, the TCA cycle intermediate is succinate.

In another aspect, the invention provides methods of treating a condition associated with altered TCA cycle metabolism in a subject. The methods include providing to a subject having a condition associated with altered TCA cycle metabolism a composition comprising one or more TCA cycle intermediates or prodrugs, analogs, or derivatives thereof formulated for non-oral administration. Preferably, the TCA cycle intermediate is succinate.

The composition may be formulated for administration by any non-oral route. For example, the composition may be formulated for injection or infusion. The injection or infusion may be subcutaneous, intravenous, intraarterial, or intramuscular. The composition may be formulated for non-oral enteral administration, such as rectal administration.

The composition may include a buffering agent that maintains a neutral or near-neutral pH of the composition. For example, the buffering agent may be present in amount sufficient to buffer the pH of the composition to from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0.

The TCA cycle intermediate may be provided at any therapeutically effective dose. For example, the TCA cycle intermediate may be provided at from about 0.1 mg/kg subject weight to about 5 g/kg subject weight, from about 0.2 mg/kg subject weight to about 5 g/kg subject weight, from about 0.5 mg/kg subject weight to about 5 g/kg subject weight, from about 1 mg/kg subject weight to about 5 g/kg subject weight, from about 2 mg/kg subject weight to about 5 g/kg subject weight, from about 5 mg/kg subject weight to about 5 g/kg subject weight, from about 0.1 mg/kg subject weight to about 2 g/kg subject weight, from about 0.2 mg/kg subject weight to about 2 g/kg subject weight, from about 0.5 mg/kg subject weight to about 2 g/kg subject weight, from about 1 mg/kg subject weight to about 2 g/kg subject weight, from about 2 mg/kg subject weight to about 2 g/kg subject weight, from about 5 mg/kg subject weight to about 2 g/kg subject weight, from about 0.1 mg/kg subject weight to about 1 g/kg subject weight, from about 0.2 mg/kg subject weight to about 1 g/kg subject weight, from about 0.5 mg/kg subject weight to about 1 g/kg subject weight, from about 1 mg/kg subject weight to about 1 g/kg subject weight, from about 2 mg/kg subject weight to about 1 g/kg subject weight, from about 5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

The condition may be any disease, disorder, or condition associated with altered TCA cycle metabolism. For example, the condition may be a disorder related to POLG mutation, an energetic disorder, glutaric acidemia type 1 or type 2, a long chain fatty acid oxidation disorder, methylmalonic acidemia (MMA), a mitochondrial associated disease, a mitochondrial encephalomyopathy lactic acidosis and stroke-like syndrome (MELAS), myoclonic epilepsy and ragged-red fibers (MERRF), mitochondrial myopathy, a mitochondrial respiratory chain deficiency, muscular dystrophy (e.g., Duchenne's muscular dystrophy and Becker's muscular dystrophy), a neurologic disorder, a pain or fatigue disease, propionic acidemia (PA), pyruvate carboxylase deficiency, refractory epilepsy, or succinyl CoA lyase deficiency. Preferably, the condition is MMA or PA.

In another aspect, the invention provides combination therapies for treating a condition associated with altered TCA cycle metabolism in a subject. The combination therapies include a non-oral formulation containing succinate or a prodrug, analog, or derivative thereof and a formulation containing citrate, citric acid, or a prodrug, analog, or derivative of citrate or citric acid.

In another aspect, the invention provides methods of treating a condition associated with altered TCA cycle metabolism in a subject. The methods include providing to a subject having a condition associated with altered TCA cycle metabolism a combination therapy including a non-oral formulation containing succinate or a prodrug, analog, or derivative thereof and a formulation containing citrate, citric acid, or a prodrug, analog, or derivative of citrate or citric acid.

The non-oral formulation containing succinate or a prodrug, analog, or derivative thereof may be formulated for administration by any non-oral means, as described above.

The non-oral formulation containing succinate or a prodrug, analog, or derivative thereof may contain a buffering agent, as described above.

The non-oral formulation containing succinate or a prodrug, analog, or derivative thereof may be provided at any therapeutically effective dose. For example, succinate or a prodrug, analog, or derivative thereof may be provided at from about 0.1 mg/kg subject weight to about 5 g/kg subject weight, from about 0.1 mg/kg subject weight to about 5 g/kg subject weight, from about 0.2 mg/kg subject weight to about 2 g/kg subject weight, from about 0.5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

The formulation containing citrate, citric acid, or a prodrug, analog, or derivative of citrate or citric acid may be formulated for administration by any suitable means. For example, the formulation may be formulated for oral, enteral, parenteral, subcutaneous, intravenous, intraarterial, or intramuscular administration. Preferably, the formulation is formulated for oral administration.

The formulation containing citrate, citric acid, or a prodrug, analog, or derivative of citrate or citric acid may contain a buffering agent. For example, the buffering agent may be present in amount sufficient to buffer the pH of the composition to from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0. The buffering agent may be an amino acid or a metal ion. The amino acid may be a natural or non-natural amino acid. The amino acid may be lysine, ornithine, or a derivative thereof.

The formulation containing citrate or a prodrug, analog, or derivative thereof may be provided at any therapeutically effective dose. For example, citrate or a prodrug, analog, or derivative thereof may be provided at from about 0.1 mg/kg subject weight to about 5 g/kg subject weight, from about 0.1 mg/kg subject weight to about 5 g/kg subject weight, from about 0.2 mg/kg subject weight to about 2 g/kg subject weight, from about 0.5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

The non-oral formulation containing succinate or a prodrug, analog, or derivative thereof, the formulation containing citrate, citric acid, or a prodrug, analog, or derivative of citrate or citric acid, or both may be provided in multiple doses per day. For example, one or more of the formulations may be provided in 2, 3, 4, 5, 6, or more doses per day.

The non-oral formulation containing succinate or a prodrug, analog, or derivative thereof and the formulation containing citrate, citric acid, or a prodrug, analog, or derivative thereof may be provided simultaneously, sequentially in either order, or in an alternating manner. Sequential administration or alternating administration may include providing the non-oral formulation containing succinate or a prodrug, analog, or derivative thereof exclusively for a period of time and providing the formulation containing citrate, citric acid, or a prodrug, analog, or derivative of citrate or citric acid exclusively for a period of time. Sequential administration may include an period of overlap in which the subject is provided both the non-oral formulation containing succinate or a prodrug, analog, or derivative thereof and the formulation containing citrate, citric acid, or a prodrug, analog, or derivative of citrate or citric acid. The periods of exclusivity and periods of overlap may independently be 1 day, 2 days, 3 days, 4 days, 5 days, 6 days, 1 week, 2 weeks, 3 weeks, 4 weeks, 6 weeks, 8 weeks, 12 weeks, 3 months, 4 months, 5 months, 6 months, 8 months, 10 months, 12 months, 18 months, or 24 months.

The condition may be any disease, disorder, or condition associated with altered TCA cycle metabolism, as described above.

In another aspect, the invention provides compositions that include a nutritional source for infants, such as baby formula or human breast milk; a non-salt form of citric acid or a prodrug, analog, or derivative thereof; and a buffering agent in an amount to buffer a pH of the composition from about 3.0 to about 8.0.

The buffering agent may be present in amount sufficient to buffer the pH of the composition to from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0. The buffering agent may be an amino acid or a metal ion. The amino acid may be a natural or non-natural amino acid. The amino acid may be lysine, ornithine, or a derivative thereof.

In another aspect, the invention provides methods of treating a condition associated with altered TCA cycle metabolism in a subject. The methods include providing to a subject having a condition associated with altered TCA cycle metabolism an oral formulation containing citrate and a buffering agent in an amount to buffer a pH of the composition from about 3.0 to about 8.0.

The buffering agent may be present in amount sufficient to buffer the pH of the composition to from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0. The buffering agent may be an amino acid or a metal ion. The amino acid may be a natural or non-natural amino acid. The amino acid may be lysine, ornithine, or a derivative thereof.

The condition may be any disease, disorder, or condition associated with altered TCA cycle metabolism, as described above. The subject may be human. The human may be a child. For example, the child may be less than 18 years in age, less than 12 years in age, less than 10 years in age, less than 8 years in age, less than 6 years in age, less than 5 years in age, less than 4 years in age, less than 3 years in age, less than 2 years in age, or less than 1 year in age. In another aspect, the invention provides methods of treating or preventing Leber's hereditary optic neuropathy in a subject. The methods include providing a subject having or at risk of developing Leber's hereditary optic neuropathy a non-oral formulation containing succinate or a prodrug, analog, or derivative thereof.

The non-oral formulation may be provided intraocularly. The non-oral formulation may be provided systemically. For example, the non-oral formulation may be provided intravenously or subcutaneously.

The non-oral formulation may include a buffering agent that maintains a neutral or near-neutral pH of the composition. For example, the buffering agent may be present in amount sufficient to buffer the pH of the composition to from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0.

The methods of treating or preventing Leber's hereditary optic neuropathy in a subject may include providing a formulation containing citrate, citric acid, or a prodrug, analog, or derivative of citrate or citric acid. The non-oral formulation and the formulation containing citrate, citric acid, or a prodrug, analog, or derivative of citrate or citric acid may be a single formulation. Alternatively, the non-oral formulation and the formulation containing citrate, citric acid, or a prodrug, analog, or derivative thereof may be separate formulations.

The formulation containing citrate, citric acid, or a prodrug, analog, or derivative of citrate or citric acid may be provided by any route of administration. For example, the formulation containing citrate, citric acid, or a prodrug, analog, or derivative of citrate or citric acid may be provided orally, intraocularly, subcutaneously, or intravenously.

The formulation containing citrate, citric acid, or a prodrug, analog, or derivative of citrate or citric acid may contain a buffering agent. For example, the buffering agent may be present in amount sufficient to buffer the pH of the composition to from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0. The buffering agent may be an amino acid or a metal ion. The amino acid may be a natural or non-natural amino acid. The amino acid may be lysine, ornithine, or a derivative thereof.

The non-oral formulation containing succinate or a prodrug, analog, or derivative thereof, the formulation containing citrate, citric acid, or a prodrug, analog, or derivative of citrate or citric acid, or both may be provided in multiple doses per day. For example, one or more of the formulations may be provided in 2, 3, 4, 5, 6, or more doses per day.

In another aspect, the invention provides formulations that contain citric acid, or a prodrug, analog, derivative thereof; one or more citrate salts, or a prodrug, analog, or derivative thereof; and an amino acid buffering agent.

The citrate salts may include monosodium citrate or monopotassium citrate.

The amino acid buffering agent may be lysine or ornithine.

The formulation may contain a sugar. The sugar may be sucrose, fructose, galactose, maltose, or lactose.

The formulation may be a powder that is soluble in an aqueous medium. The formulation may be an aqueous solution.

The formulation, or the soluble components within the formulation, may contain citric acid, monosodium citrate, monopotassium citrate, lysine, and sucrose. The formulation may contain, by mass, from about 40% to about 60% citric acid; from about 1% to about 10% monosodium citrate; from about 0.1% to about 5% monopotassium citrate; from about 30% to about 40% lysine; and from about 10% to about 15% sucrose. The formulation, or the soluble components within the formulation, may contain, by mass, about 49.2% citric acid, about 2% monosodium citrate, about 0.3% monopotassium citrate, about 37.5% lysine, and about 11% sucrose. The formulation, or the soluble components within the formulation, may contain, by mass, about 44.2% citric acid, about 8.3% monosodium citrate, about 2.3% monopotassium citrate, about 33.8% lysine, and about 11.5% sucrose. The formulation, or the soluble components within the formulation, may contain, by mass, from about 50% to about 60% citrate, citric acid, or a combination thereof; from about 0.2% to about 1% sodium; from about 0.02% to about 0.5% potassium; from about 30% to about 40% lysine; and from about 10% to about 15% sucrose.

The formulation may be suitable for oral administration.

The formulation may be given to a subject to treat or prevent a condition associated with altered TCA cycle metabolism, such as any of those described above.

In another aspect, the invention provides a formulation including citric acid, or a prodrug, analog, derivative thereof; at least one citrate salt, or a prodrug, analog, or derivative thereof; and an amino acid. The formulation may include citric acid and at least one citrate salt, for example, the at least one citrate salt may be selected from the group consisting of monosodium citrate and monopotassium citrate. In other embodiments, the formulation may be citric acid and at least two citrate salts, for example, the at least two citrate salts may be monosodium citrate and monopotassium citrate.

The formulation may include any natural or non-natural amino acid. In certain embodiments, the amino acid is a cationic amino acid, such as lysine or arginine. The formulation may include other components, such as a sugar, for example sucrose.

The formulation may be provided by any route of administration, and a preferable route is oral. An exemplary oral formulation is a powder that is soluble in an aqueous medium. In a particular embodiment, the formulation includes citric acid, monosodium citrate, monopotassium citrate, sucrose, and one or more of lysine and arginine.

Other aspects of the invention provide methods of treating a condition associated with altered TCA cycle metabolism in a subject. Such methods may involve providing to a subject having a condition associated with altered TCA cycle metabolism a formulation comprising (i) citric acid, or a prodrug, analog, derivative thereof; (ii) at least one citrate salt, or a prodrug, analog, or derivative thereof; and (iii) an amino acid, wherein a therapeutic effect is achieved in the subject by activity of a combination of two or more of components (i), (ii), and (iii) in the formulation. In certain embodiments, the amino acid is incorporated into the TCA cycle of the subject and contributes to the therapeutic effect that is achieved in the subject.

Exemplary conditions include a disorder related to POLG mutation, an energetic disorder, glutaric acidemia type 1 or type 2, a long chain fatty acid oxidation disorder, methylmalonic acidemia (MMA), a mitochondrial associated disease, a mitochondrial encephalomyopathy lactic acidosis and stroke-like syndrome (MELAS), myoclonic epilepsy and ragged-red fibers (MERRF), mitochondrial myopathy, a mitochondrial respiratory chain deficiency, muscular dystrophy (e.g., Duchenne's muscular dystrophy and Becker's muscular dystrophy), a neurologic disorder, a pain or fatigue disease, propionic acidemia (PA), pyruvate carboxylase deficiency, refractory epilepsy, or succinyl CoA lyase deficiency.

The formulation may include citric acid and at least one citrate salt, for example, the at least one citrate salt may be selected from the group consisting of monosodium citrate and monopotassium citrate. In other embodiments, the formulation may be citric acid and at least two citrate salts, for example, the at least two citrate salts may be monosodium citrate and monopotassium citrate.

The formulation may include any natural or non-natural amino acid. In certain embodiments, the amino acid is a cationic amino acid, such as lysine or arginine. The formulation may include other components, such as a sugar, for example sucrose.

The formulation may be provided by any route of administration, and a preferable route is oral. An exemplary oral formulation is a powder that is soluble in an aqueous medium. In a particular embodiment, the formulation includes citric acid, monosodium citrate, monopotassium citrate, sucrose, and one or more of lysine and arginine. The formulation may be provided in one or multiple doses per day. For example, one or more of the formulations may be provided in 1, 2, 3, 4, 5, 6, or more doses per day.

The formulation containing citrate or a prodrug, analog, or derivative thereof may be provided at any therapeutically effective dose. For example, citrate or a prodrug, analog, or derivative thereof may be provided at from about 0.1 mg/kg subject weight to about 5 g/kg subject weight, from about 0.1 mg/kg subject weight to about 5 g/kg subject weight, from about 0.2 mg/kg subject weight to about 2 g/kg subject weight, from about 0.5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

The formulation, or the soluble components within the formulation, may contain citric acid, monosodium citrate, monopotassium citrate, lysine, and sucrose. The formulation may contain, by mass, from about 40% to about 60% citric acid; from about 1% to about 10% monosodium citrate; from about 0.1% to about 5% monopotassium citrate; from about 30% to about 40% lysine; and from about 10% to about 15% sucrose. The formulation, or the soluble components within the formulation, may contain, by mass, about 49.2% citric acid, about 2% monosodium citrate, about 0.3% monopotassium citrate, about 37.5% lysine, and about 11% sucrose. The formulation, or the soluble components within the formulation, may contain, by mass, about 44.2% citric acid, about 8.3% monosodium citrate, about 2.3% monopotassium citrate, about 33.8% lysine, and about 11.5% sucrose. The formulation, or the soluble components within the formulation, may contain, by mass, from about 50% to about 60% citrate, citric acid, or a combination thereof; from about 0.2% to about 1% sodium; from about 0.02% to about 0.5% potassium; from about 30% to about 40% lysine; and from about 10% to about 15% sucrose.

### BRIEF DESCRIPTION OF THE DRAWINGS

FIG. 1 is a graph of plasma concentration of ¹³C₄-succinate at various time points in individual rats after subcutaneous administration of ¹³C₄-succinate at 10 mg/kg.

FIG. 2 is a graph of average plasma concentration of ¹³C₄-succinate at various time points in rats after subcutaneous administration of ¹³C₄-succinate at 10 mg/kg.

FIG. 3 is a graph of plasma concentration of ¹³C₄-succinate at various time points in individual rats after subcutaneous administration of ¹³C₄-succinate at 50 mg/kg.

FIG. 4 is a graph of average plasma concentration of ¹³C₄-succinate at various time points in rats after subcutaneous administration of ¹³C₄-succinate at 50 mg/kg.

FIG. 5 is a graph of plasma concentration of ¹³C₄-succinate at various time points in individual rats after subcutaneous administration of ¹³C₄-succinate at 100 mg/kg.

FIG. 6 is a graph of average plasma concentration of ¹³C₄-succinate at various time points in rats after subcutaneous administration of ¹³C₄-succinate at 100 mg/kg.

FIG. 7 is graph showing the effects of citrate and succinate on basal respiration in cells from a patient with propionic acidemia (PA).

FIG. 8 is a graph of plasma concentration of ¹³C₄-succinate at various time points in individual rats after intravenous administration of ¹³C₄-succinate at 10 mg/kg.

FIG. 9 is a graph of average plasma concentration of ¹³C₄-succinate at various time points in rats after intravenous administration of ¹³C₄-succinate at 10 mg/kg.

FIG. 10 is a graph of plasma concentration of ¹³C₄-succinate at various time points in individual rats after oral administration of ¹³C₄-succinate at 50 mg/kg.

FIG. 11 is a graph of average plasma concentration of ¹³C₄-succinate at various time points in rats after oral administration of ¹³C₄-succinate at 50 mg/kg.

FIG. 12 is a graph of plasma concentration of ¹³C₆-citrate at various time points in individual rats after intravenous administration of ¹³C₆-citrate at 10 mg/kg.

FIG. 13 is a graph of average plasma concentration of ¹³C₆-citrate at various time points in rats after intravenous administration of ¹³C₆-citrate at 10 mg/kg.

FIG. 14 is a graph of plasma concentration of ¹³C₆-Citrate at various time points in individual rats after oral administration of ¹³C₆-Citrate at 50 mg/kg.

FIG. 15 is a graph of average plasma concentration of ¹³C₄-succinate at various time points in rats after oral administration of ¹³C₆-Citrate at 50 mg/kg.

### DETAILED DESCRIPTION

The invention provides compositions and methods that allow efficient delivery of intermediates of the TCA cycle for treatment of metabolic disorders. In particular, the compositions and methods of the invention enable delivery of succinate by subcutaneous or intravenous administration. Synthesis of succinate is defective in propionic acidemia and methylmalonic acidemia, and the errant metabolism in patients with these disorders lead to a variety of potentially fatal effects, such as neurological damage, cardiomyopathy, and infections. The non-oral formulations of succinate permit delivery of the compound with much higher bioavailability that can be achieved with prior oral succinate formulations. Consequently, the non-oral formulations provide succinate at sufficient levels to prevent serious, long-term effects of defective TCA cycle metabolism.

The invention also provides combination therapies that include non-oral formulations of succinate and oral formulations of citrate, another intermediate of the TCA cycle. In contrast to succinate, citrate can administered orally at therapeutically effective doses. The combination therapies thus provide delivery of different TCA cycle intermediates by different means. The non-oral formulation of succinate and oral formulation of citrate may be provided sequentially or simultaneously. Sequential combination therapies allow patients, particularly infants, to transition from non-oral therapies to oral administration when they become old enough to ingest citrate in therapeutically effective quantities. Such therapies allow early intervention in newborns while permitting long-term care that can be administered without a health-care professional. Simultaneous combination therapies are useful when clinical issues, such as vomiting, skin rashes, etc., limit the amount of intermediate that can be delivered by either mode of administration.

### TCA Cycle and Associated Disorders

The TCA cycle is illustrated below:

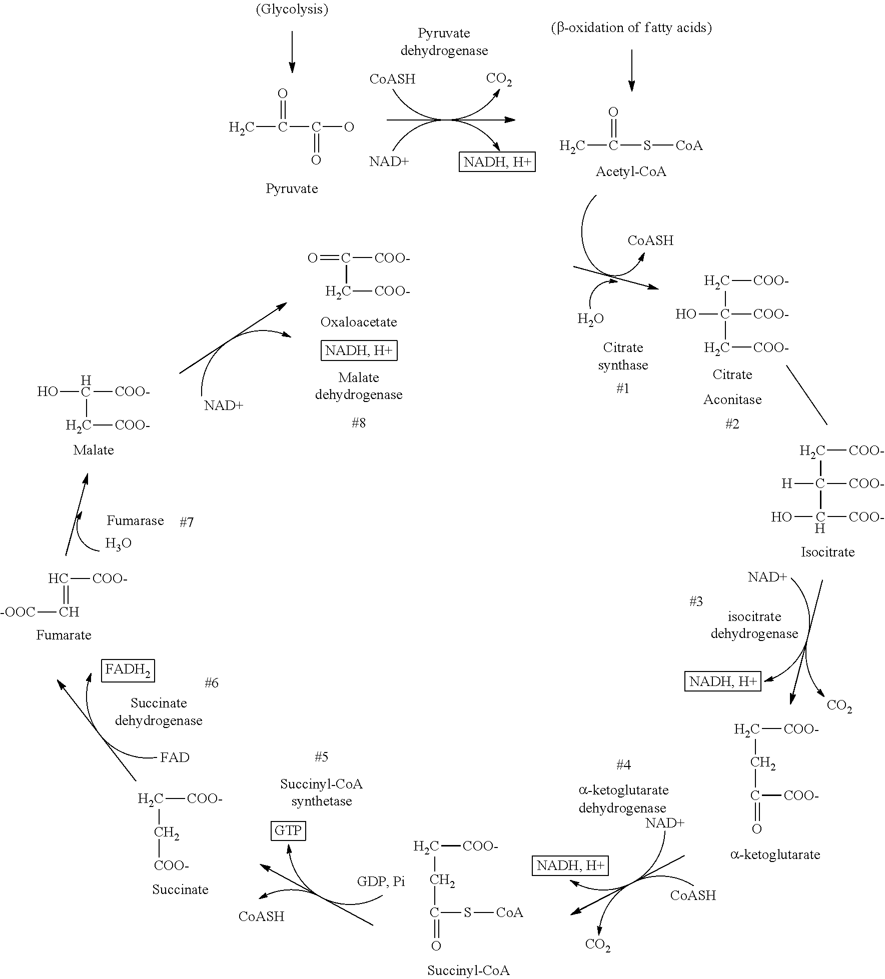

Abnormal TCA cycle metabolism is associated with a variety of conditions. In hereditary metabolic disorders of the TCA cycle, such as 2-oxoglutaric aciduria, fumarase deficiency, and succinyl-CoA synthetase deficiency, genetic mutations affect enzymes of the TCA cycle or enzymes that catalyze related reactions. Consequently, individual reactions of the TCA cycle are impaired, leading to the depletion of intermediates required for the cycle to proceed. Such diseases typically present early with severe symptoms, such as mental retardation, microcephaly, deafness, and hypotonia and are often fatal in early childhood.

Other metabolic disorders affect pathways in which other metabolites, such as fatty acids and amino acids, are converted into intermediates of the TCA cycle. For example, propionyl-CoA is generated by oxidation of odd-chain fatty acids and breakdown of the amino acids isoleucine, valine, threonine, and methionine and is then converted to succinyl-CoA. The conversion of propionyl-CoA to succinyl-CoA involves the following sequence of reactions:

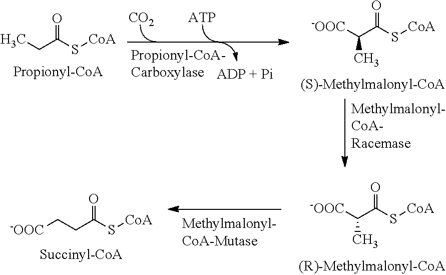

Propionic acidemia (PA) results from a deficiency in propionyl-CoA carboxylase. In patients with PA, excess propionyl-CoA is converted to propionic acid, which accumulates in the bloodstream. In an analogous manner, methylmalonic acidemia (MMA) results from a deficiency in methylmalonyl CoA mutase. In patients with MMA, excess methylmalonyl-CoA is converted to methylmalonic acid, which accumulates in the bloodstream. In both PA and MMA, the high levels of acid in the blood lead to a variety of potentially fatal effects, such as organ damage, stroke, and seizure.

Treatment of PA and MMA focuses on dietary management to avoid amino acids that trigger acid accumulation. Consequently, patients with PA or MMA must strictly adhere to a low-protein diet to minimize their intake of triggering amino acids. The restricted intake of amino acids in patients with PA or MMA often leads to a shortage of L-carnitine, a quaternary ammonium compound involved in transport of fatty acids across the mitochondrial membrane. Consequently, patients with PA or MMA typically receive supplemental L-carnitine. The low-protein diet of infants with PA or MMA also puts them at risk for bacterial infections, so they may be given antibiotics prophylactically.

PA and MMA are autosomal recessive genetic disorders. Homozygous mutations in either of PCCA or PCCB, the genes that encode propionyl-CoA carboxylase, cause PA. MMA can result from homozygous mutations in MUT, which encodes methylmalonyl-CoA mutase. Methylmalonyl-CoA mutase requires vitamin B₁₂ as a cofactor, and mutations in genes involved in vitamin B₁₂ metabolism, such as LMBRD1, MCEE, MMAA, MMAB, MMACHC, and MMADHC, can also cause MMA. A dietary deficiency of vitamin B₁₂ can also lead to MMA.

Abnormal TCA cycle metabolism is also observed in other diseases that do not have direct genetic links to this metabolic pathway. For example, altered TCA metabolism is observed in neurodegenerative disorders, such as Amyotrophic Lateral Sclerosis, Alzheimer's disease, Parkinson's disease, or Huntington's disease, and in a wide variety of cancers. Although the symptoms this diverse set of diseases vary, in many cases decreased activity of specific TCA enzymes or decreased mitochondrial ATP production has been observed, and it is believed that boosting levels of TCA cycle intermediates would mitigate the symptoms and improve prognoses.

The invention provides methods for treating any disease, disorder, or condition associated with altered TCA cycle metabolism or that can be ameliorated by providing an intermediate of the TCA cycle. The condition may be an inherited disorder, such as PA, MMA, 2-oxoglutaric aciduria, fumarase deficiency, or succinyl-CoA synthetase deficiency. The condition may be a neurodegenerative disorder, such as Amyotrophic Lateral Sclerosis, Alzheimer's disease, Parkinson's disease, or Huntington's disease. The condition may be a cancer, such as pancreatic cancer, kidney cancer, cervical cancer, prostate cancer, muscle cancer, gastric cancer, colon cancer, glioblastoma, glioma, paraganglioma, leukemia, liver cancer, breast cancer, carcinoma, and neuroblastoma.

Additional metabolic diseases, disorders, or conditions that can be treated with compositions or methods of the invention include acute angina, acute kidney injury, acute starvation, adrenoleukodystrophy (ALD), adrenomyeloneuropathy (AMN), an age-associated disease, Alpers disease (progressive infantile poliodystrophy), Alzheimer's disease, amyotrophic lateral sclerosis (ALS), atrial fibrillation, autism and autism spectrum disorders (ASD), Barth syndrome (lethal infantile cardiomyopathy), beta-oxidation defects, bioenergetic metabolism deficiency, bipolar disorder, carnitine-acyl-carnitine deficiency, carnitine deficiency, carnitine palmitoyltransferase I (CPT I) deficiency, carnitine palmitoyltransferase II (CPT II) deficiency, a cerebral vascular accident, chronic progressive external ophthalmoplegia syndrome) (CPEO), coenzyme Q10 deficiency, complex I deficiency (NADH dehydrogenase (NADH¬CoQ reductase) deficiency), complex II deficiency (succinate dehydrogenase deficiency), complex III deficiency (ubiquinone-cytochrome c oxidoreductase deficiency, complex IV deficiency/COX deficiency, complex V deficiency (ATP synthase deficiency), coronary occlusion, COX deficiency, creatine deficiency syndromes (e.g., cerebral creatine deficiency syndromes (CCDS), guanidinoaceteate methyltransferase deficiency (GAMT deficiency), L-arginine: glycine amidinotransferase deficiency (AGAT deficiency), and SLC6A8-related creatine transporter deficiency (SLC6A8 deficiency)), diabetes, disorders related to POLG mutations, endotoxemia, an energetic disorder, epilepsy, Friedreich's ataxia (FRDA or FA), glutaric aciduria type II, Huntington's disease, hypoxia, ischemia, Kearns-Sayre syndrome (KSS), lactic acidosis, long-chain acyl-CoA dehydrogenase deficiency) (LCAD, VLCAD, VLCADD), long-chain 3-hydroxyacyl-CoA dehydrogenase deficiency (LCHAD), Leigh disease or syndrome (subacute necrotizing encephalomyelopathy), Leber's hereditary optic neuropathy (LHON), Luft disease, macular degeneration, male infertility, medium-chain acyl-CoA dehydrogenase deficiency (MCAD), mitochondrial associated disease, mitochondrial cytopathy, mitochondrial encephalomyopathy lactic acidosis and stroke-like syndrome (MELAS), mitochondrial encephalopathy (including encephalomyopathy, encephalomyelopathy), mitochondrial DNA depletion, mitochondrial myopathy, mitochondrial recessive ataxia syndrome (MIRAS), mitochondrial respiratory chain deficiency, multiple organ dysfunction syndrome, mood disorders, motor neuron diseases, muscular dystrophy (e.g., Duchenne's muscular dystrophy and Becker's muscular dystrophy), myocardial infarction, myoclonic epilepsy and ragged-red fibers (MERRF), myoneurogastointestinal disorder and encephalopathy (MNGIE), neuropathy, ataxia, and retinitis pigmentosa (NARP), neurodegenerative disorders associated with Parkinson's, Pearson syndrome, pyruvate carboxylase deficiency, pyruvate dehydrogenase deficiency, refractory epilepsy, renal tubular acidosis, respiratory chain deficiencies, schizophrenia, sepsis, short-chain acyl-CoA dehydrogenase deficiency (SCAD), short-chain hydroxyacyl-CoA dehydrogenase deficiency (SCHAD, SCHADD), stroke, succinyl CoA lyase deficiency, systemic inflammatory response syndrome, and very long-chain acyl-CoA dehydrogenase deficiency (VLCAD).

The disease, disorder, or condition may be secondary to, or associated with another disease, disorder, or condition. For example, the disease disorder or condition may be associated with cardiac disease, a gastrointestinal disorder, ischemia reperfusion injury, intoxication, liver disease, loss of motor control, muscle weakness and pain, mutations in the mitochondrial genome, a neurologic disease, disorder or condition, pain or fatigue disease, seizures, sensineural hearing loss, swallowing difficulties, tinnitus, or visual/hearing problems.

Diseases, disorders, and conditions associated with altered TCA cycle metabolism often affect organs, tissues, and systems that have high energy demands, such as the brain, cochlea, endocrine system, heart, kidney, liver, respiratory system, retina, and skeletal muscles. Thus, the compositions and methods of the invention may be used to treat diseases, disorders, or conditions that affect one or more of these organs, tissues, or systems.

Another clinically important metabolic pathway is the mitochondrial electron transport chain. The electron transport chain uses a complex series of redox reactions to create a proton gradient across the mitochondrial inner membrane, and the chemiosmotic potential from the proton gradient is used to drive adenosine triphosphate (ATP) synthesis. The electron transport chain involves four enzymatic complexes in the mitochondrial inner membrane: NADH dehydrogenase, also called respiratory complex I; succinate dehydrogenase, also called respiratory complex II; coenzyme Q:cytochrome c reductase, also called respiratory complex III; and cytochrome c oxidase, also called respiratory complex IV. Electrons enter the transport chain in either of two ways. First, NADH dehydrogenase may transfer electrons from NADH to ubiquinone, the first intermediate electron carrier in the chain. Alternatively, electrons from succinate may be transferred to ubiquinone by succinate dehydrogenase. In the next step of the electron transport chain, electrons are transferred from ubiquinone to cytochrome c, the second intermediate electron carrier, by coenzyme Q:cytochrome c reductase. In the final step, cytochrome c oxidase transfers electrons from cytochrome c to molecular oxygen to form water, the net product of electron transport. Succinate dehydrogenase is the only enzyme that participates in both the TCA cycle and the electron transport chain.

Leber's hereditary optic neuropathy (LHON) is a retinal degenerative condition caused by defects in the electron transport chain. LHON results from mutations in mitochondrial genes that encode components of NADH dehydrogenase, such as MT-ND1, MT-ND4, MT-ND4L, and MT-ND6. Because mutations that cause LHON are encoded by genes in the mitochondrial genome, which is transmitted to the embryo from the egg but not from the sperm, LHON can only be inherited maternally.

An insight of the invention is that succinate is useful for treatment of LHON. Due to reduced NADH dehydrogenase activity, electron transport and ATP synthesis are decreased in patients with LHON. In particular, formation of ubiquinone, the first intermediate electron carrier in the chain, is diminished. However, electron transport activity can be restored by providing supplemental succinate, which can donate electrons to form ubiquinone via succinate dehydrogenase. Thus, providing additional succinate to serve as electron donor for the electron transport chain in patients with LHON compensates for the insufficiency of electron transfer from NADH.

### Compositions Containing TCA Cycle Intermediates

The invention provides compositions that contain one or more TCA cycle intermediates or prodrugs, analogs, or derivatives thereof formulated for non-oral administration. For example and without limitation, the TCA cycle intermediate or prodrug, analog, or derivative thereof may be citrate, cis-aconitate, D-isocitrate, α-ketoglutarate, succinate, fumarate, malate, oxaloacetate, acetone, acetoacetate, β-hydroxybutyrate, β-ketopentanoate, or β-hydroxypentanoate. Preferably, the TCA cycle intermediate is succinate. The TCA cycle intermediate or prodrug, analog, or derivative thereof may be provided as a free acid, salt, or a non-salt form.

The compositions containing one or more TCA cycle intermediates or prodrugs, analogs, or derivatives thereof may be formulated for administration by any non-oral route. For example and without limitations, the compositions may be formulated for subcutaneous, intravenous, intraarterial, intramuscular, intradermal, or rectal administration. Preferably, the compositions are formulated for intravenous or subcutaneous administration.

A prodrug is a medication or compound that, after administration, is metabolized (i.e., converted within the body) into a pharmacologically active drug. The prodrug itself may be pharmacologically inactive. Prodrugs may be used to improve how a medicine is absorbed, distributed, metabolized, and excreted. The prodrug may improve the bioavailability of the active drug when the active drug is poorly absorbed from the gastrointestinal tract. The prodrug may improve how selectively the drug interacts with cells or processes that are not its intended target, thereby reducing unintended and undesirable side effects. The prodrug may be converted into a biologically active form (bioactivated) inside cells (a Type I prodrug) or outside cells (a Type II prodrug). The prodrug may bioactivated in the gastrointestinal tract, in systemic circulation, in metabolic tissue other than the target tissue, or in the target tissue.

TCA cycle intermediates or prodrugs, analogs, or derivatives thereof may be provided as pharmaceutically acceptable salts, such as nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphor sulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counter ions such as halide, hydroxide, carboxylate, sulfate, phosphate, nitrate, alkyl having from 1 to 6 carbon atoms, sulfonate and aryl sulfonate.

Prodrugs of succinate are known in the art and described in, for example, International Publication Nos. WO 1997/047584, WO 2014/053857, WO 2015/155230, WO 2015/155231, WO/2015/155238, WO 2017/060400, WO 2017/060418, and WO 2017/060422; European Patent Publication Nos. EP 2903609, EP 3129016, EP 3129364, EP 3129058, and EP 3391941; and U.S. Patent Publication Nos. US 2017/0100359, US 2017/0105960, and US 2017/0105961, the contents of each of which are incorporated herein by reference. Representative compounds from these documents are provided below.

Compounds of formulas (I) and (IA):

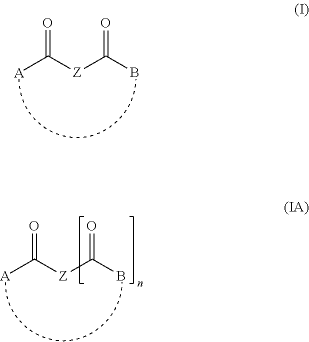

or a pharmaceutically acceptable salt thereof, wherein the dotted bond between A and B denotes an optional bond so as to form a ring closed structure,

and wherein

when the formula is Formula (I), Z is selected from —CH₂— (e.g., derived from malonic acid), —CH₂—CH₂—CH₂ (e.g., derived from glutaric acid), —CH═CH₂— (e.g., derived from fumaric acid), —CH₂—CH(OH)— (e.g., derived from malic acid), —CH(OH)—CH₂— (e.g., derived from malic acid), CH₂C(OH)(COOH)—CH₂— (e.g., derived from citric acid), —C(O)—CH₂—CH₂— (e.g., derived from alphaketoglutaric acid), —CH₂—CH₂—C(O)— (e.g., derived from alpha-ketoglutaric acid), —CH₂—C(COC(OH)—C(COOH)—CHOH)═CH— (e.g., derived from aconitic acid), —CH═C(COOH)—CH₂— (e.g., derived from aconitic acid), —CH(OH)—CH(COOH)—CH₂— (e.g., derived from isocitric acid), —CH₂—CH(COOH)—CH(OH)— (e.g., derived from isocitric acid), —CH₂—CH(COOH)—C(═O)— (e.g., derived from oxalosuccinic acid), —C(═O)—CH(COOH)—CH₂— (e.g., derived from oxalosuccinic acid), —C(═O)—CH₂— (e.g., derived from oxaloacetatic acid), —CH₂—C(═O)— (e.g., derived from oxaloacetic acid); or

when the formula is Formula (IA), Z is selected from —CH(OH)—CH₂(OH) and n is 0 e.g., derived from glyceric acid); or Z is absent or —CH₂— and n is 1 and B is an alkyl group (e.g., derived from pyruvic acid or acetoacetic acid, respectively);

A and B are independently different or the same and are selected from —OR, —OR′, —NHR″, —SR′″

or —OH; wherein R is

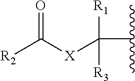

or in those cases, where the compound is according to Formula (IA), then B is C₁-C₄-alkyl, branched or straight, preferably R is Me;

R′ is selected from the formula (II), (V) or (IX) below:

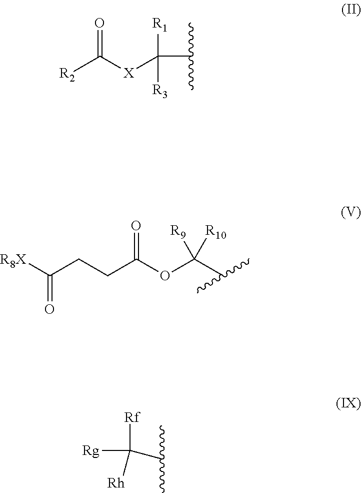

and both A and B are not —OH,

R′, R″ and R′″ are independently different or identical and are selected from formula (VII-VIII) below:

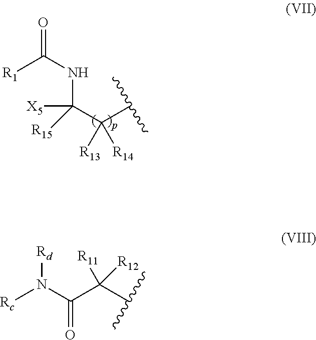

R₁ and R₃ are independently different or identical and are selected from H, Me, Et, propyl, i-propyl, butyl, iso-butyl, t-butyl, O-acyl, O-alkyl, N-acyl, N-alkyl, Xacyl, CH₂Xalkyl, CH₂CH₂CH₂OC(—O)CH₂CH₂COX₆R₈ or

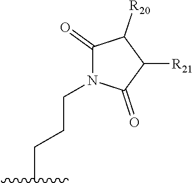

X is selected from O, NH, NR₆, S,

R₂ is selected from Me, Et, propyl, i-propyl, butyl, iso-butyl, t-butyl, C(O)CH₃, C(O)CH₂C(O)CH₃, C(O)CH₂CH(OH)CH₃,

p is an integer and is 1 or 2,

R₆ is selected from H, alkyl, Me, Et, propyl, i-propyl, butyl, iso-butyl, t-butyl, acetyl, acyl, propionyl, benzoyl, or formula (II), or formula (VIII)

X₅ is selected from —H, —COOH, —C(═O)XR₆, CONR₁R₃ or one of the formulas

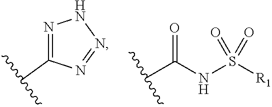

R9 is selected from H, Me, Et or O₂CCH₂CH₂COXR₈,

R₁₀ is selected from Oacyl, NHalkyl, NHacyl, or O₂CCH₂CH₂CO X₆R₈, X₆ is O or NR₈, and R₈ is selected from H, alkyl, Me, Et, propyl, i-propyl, butyl, iso-butyl, t-butyl, acetyl, acyl, propionyl, benzoyl, succinyl, or formula (II), or formula (VIII),

R₁₁ and R₁₂ are independently the same or different and are selected from H, alkyl, Me, Et, propyl, i-propyl, butyl, iso-butyl, t-butyl, acetyl, acyl, propionyl, benzoyl, succinyl, acyl, —CH₂Xalkyl, —CH₂Xacyl, where X is selected from O, NR₆ or S,

R₁₃, R₁₄ and R₁₅ are independently different or identical and are selected from H, Me, Et, propyl, i-propyl, butyl, iso-butyl, t-butyl, —COOH, O-acyl, O-alkyl, N-acyl, N-alkyl, Xacyl, CH₂Xalkyl

R_{c} and R_{d} are independently CH₂Xalkyl, CH₂Xacyl, where X═O, NR₆ or S, and alkyl is e.g. H, Me, Et, propyl, i-propyl, butyl, iso-butyl, t-butyl, and acyl is e.g. formyl, acetyl, propionyl, isopropionyl, byturyl, tert-butyryl, pentanoyl, benzoyl, succinyl, or the like,

R_{f}, R_{g}, and Rₕ are independently selected from Xacyl, —CH₂Xalkyl, —CH₂X-acyl and R9,

alkyl is selected from methyl, ethyl, propyl, isopropyl, n-butyl, isobutyl, sec-butyl, tert-butyl, n-pentyl, neopentyl, isopentyl, hexyl, isohexyl, heptyl, octyl, nonyl ordecyl, and acyl is selected from formyl, acetyl, propionyl, butyryl pentanoyl, benzoyl, succinyl and the like,

R₂₀ and R₂₁ are independently different or identical and are selected from H, lower alkyl, i.e. C₁-C₄ alkyl or R₂₀ and R₂₁ together may form a C₄-C₇ cycloalkyl or an aromatic group, both of which may optionally be substituted with halogen, hydroxyl or a lower alkyl, or

R₂₀ and R₂₁ may be

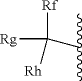

or

CH₂X-acyl, F, CH₂COOH, CH₂CO₂alkyl, and

when there is a cyclic bond present between A and B the compound is

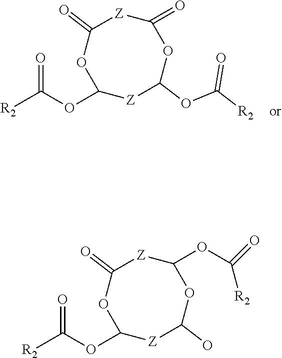

and acyls and alkyls may be optionally substituted.

Compounds of formulas (I) and (IA), or a pharmaceutically acceptable salt thereof, wherein the dotted bond between A and B denotes an optional bond so as to form a ring closed structure, and

wherein A is —OR₁, and R₁ is H or a pharmaceutically acceptable salt, or an optionally substituted alkyl group or a group of formula (II) and B in formula (I) is —OR₂ and R₂ is independently a group according to formula (II) where formula (II) is

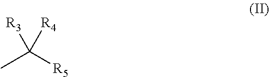

wherein R₃ and R₄ are independently H, optionally substituted C₁-C₃ alkyl, or are linked together to form a ring and where R₅ is linked to R₁ to form a ring or R₅ is selected from OCORₐ, OCOOR_{b}, OCONR_{c}R_{d}, SO₂Rₑ, OPO(OR_{f})(OR_{g}) or CONR_{c}R_{d} where Rₐ is optionally substituted alkyl or optionally substituted cycloalkyl, R_{b} is optionally substituted alkyl, R_{c} and R_{d} are independently H, optionally substituted alkyl or are linked together to form a ring which may contain one or more further heteroatoms, Rₑ is optionally substituted alkyl, R_{f} and R_{g} are independently, H, methyl, ethyl or are linked together to form a ring;

wherein B in Formula (IA) is C₁-C₄ alkyl, branched or straight;

and wherein

when the formula is Formula (I), Z is selected from —CH₂— (e.g., derived from malonic acid), —CH₂—CH₂—CH₂ (e.g., derived from glutaric acid), —CH═CH₂— (e.g., derived from fumaric acid), —CH₂—CH(OH)— (e.g., derived from malic acid), —CH(OH)—CH₂— (e.g., derived from malic acid), CH₂C(OH)(COOH)—CH₂— (e.g., derived from citric acid —C(O)—CH₂—CH₂— (e.g., derived from alpha-ketoglutaric acid), —CH₂—CH₂—C(O)— (e.g., derived from alpha-ketoglutaric acid), —CH₂—C(COC(OH)—C(COOH)—CHOH)═CH— (e.g., derived from aconitic acid), —CH═C(COOH)—CH₂— (e.g., derived from aconitic acid), —CH(OH)— CH(COOH)—CH₂— (e.g., derived from isocitric acid), —CH₂—CH(COOH)—CH(OH)— (e.g., derived from isocitric acid), —CH₂—CH(COOH)—C(═O)— (e.g., derived from oxalosuccinic acid), —C(═O)—CH(COOH)—CH₂— (e.g., derived from oxalosuccinic acid), —C(═O)—CH₂— (e.g., derived from oxaloacetatic acid), —CH₂—C(═O)— (e.g., derived from oxaloacetic acid); or

when the formula is Formula (IA), Z is selected from —CH(OH)—CH₂(OH) and n is 0 e.g., derived from glyceric acid); or Z is absent or —CH₂— and n is 1 and B is an alkyl group (e.g., derived from pyruvic acid or acetoacetic acid, respectively).

Compounds of formulas (I) and (IA), or a pharmaceutically acceptable salt thereof, wherein the dotted bond between A and B denotes an optional bond so as to form a ring closed structure,

and wherein

when the formula is Formula (I), Z is selected from —CH₂— (e.g., derived from malonic acid), —CH₂—CH₂—CH₂ (e.g., derived from glutaric acid), —CH═CH₂— (e.g., derived from fumaric acid), —CH₂—CH(OH)— (e.g., derived from malic acid), —CH(OH)—CH₂— (e.g., derived from malic acid), CH₂C(OH)(COOH)—CH₂— (e.g., derived from citric acid), —C(O)—CH₂—CH₂— (e.g., derived from alphaketoglutaric acid), —CH₂—CH₂—C(O)— (e.g., derived from alpha-ketoglutaric acid), —CH₂—C(COC(OH)—C(COOH)—CHOH)═CH— (e.g., derived from aconitic acid), —CH═C(COOH)—CH₂— (e.g., derived from aconitic acid), —CH(OH)—CH(COOH)—CH₂— (e.g., derived from isocitric acid), —CH₂—CH(COOH)—CH(OH)— (e.g., derived from isocitric acid), —CH₂—CH(COOH)—C(═O)— (e.g., derived from oxalosuccinic acid), —C(═O)—CH(COOH)—CH₂— (e.g., derived from oxalosuccinic acid), —C(═O)—CH₂— (e.g., derived from oxaloacetatic acid), —CH₂—C(═O)— (e.g., derived from oxaloacetic acid); or

when the formula is Formula (IA), Z is selected from —CH(OH)—CH₂(OH) and n is 0 e.g., derived from glyceric acid); or Z is absent or —CH₂— and n is 1 and B is an alkyl group (e.g., derived from pyruvic acid or acetoacetic acid, respectively);

A is selected from —SR, —OR and NHR, and R is

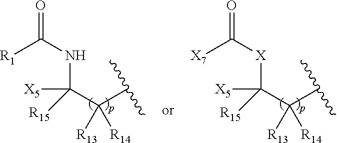

when Z is CH₂ and R is Me, Et, propyl, butyl, pentyl, hexyl, heptyl, octyl or succinyl, then X₅, R₁₅, R₁₄ and R₁₃ cannot all be H;

when Z is —CH₂—CH₂—CH₂, —CH═CH—, —CH₂—CH(OH)—, —CH(OH)—CH₂—, —CH₂C(OH)(COOH)—CH₂—, —C(O)—CH₂—CH₂—, —CH₂—CH₂—C(O)—, —CH₂—C(COOH)═CH—, —CH═C(COOH)—CH₂—, —CH(OH)—CH(COOH)—CH2-, —CH2-CH(COOH)—CH(OH)—, —CH2—CH(COOH)—C(═O)—, —C(═O)—CH(COOH)—CH2-, —C(═O)—CH2-, or —CH2-C(═O)— and R₁ is Me, octyl or succinyl, then X₅, R₁₅, R₁₄ and R₁₃ cannot all be H;

R₁ cannot contain the motif —CH2CH2N-acyl; R₁ cannot be glutamate; when in formula (IA), Z is —CH(OH)—CH2(OH) and n is 0, or Z is absent and n is 1 and B is an alkyl group (e.g., derived from pyruvic acid) then R₁ cannot be Me when X₅ is —COOH, or —C(═O)XR₆;

B is selected from —O—R′, —NHR″, —SR′″ or —OH; and R′ is selected from the formula (II) to (IX) below:

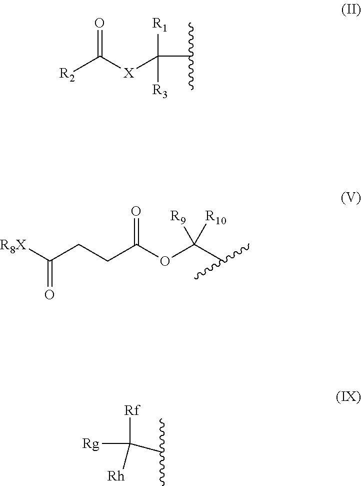

or in those cases, where the compound is according to Formula (IA), then B is C₁-C₄-alkyl, branched or straight, preferably R is Me;

R′, R″ and R′″ are independently different or identical and is selected from formula (VII-VIII) below:

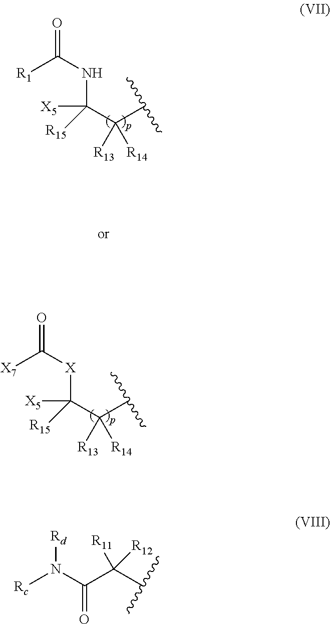

R₁ and R₃ are independently different or identical and are selected from H, Me, Et, propyl, i-propyl, butyl, iso-butyl, t-butyl, O-acyl, O-alkyl, N-acyl, N-alkyl, Xacyl, —CH₂Xalkyl, —CH₂X-acyl, F, —CH₂COOH, —CH₂CO₂alkyl,

X is selected from O, NH, NR₆, S,

R₂ is selected from Me, Et, propyl, i-propyl, butyl, iso-butyl, t-butyl, —C(O)CH₃, —C(O)CH₂C(O)CH₃, —C(O)CH₂CH(OH)CH₃,

p is an integer and is 1 or 2

R₆ is selected from H, Me, Et, propyl, i-propyl, butyl, iso-butyl, t-butyl, acetyl, acyl, propionyl, benzoyl, or formula (II), or formula (VIII)

X₅ is selected from —H, Me, Et, propyl, i-propyl, butyl, iso-butyl, t-butyl, —COOH, —C(═O)XR₆, CONR₁R₃ or is formula

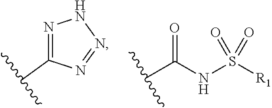

X7 is selected from R₁, —NR₁R₃,

R9 is selected from H, Me, Et or O₂CCH₂CH₂COXR₈

R₁₀ is selected from —Oacyl, —NHalkyl, —NHacyl, or O₂CCH₂CH₂COX₆R₈

X₆ is selected from O, NR₈, NR₆R₈, wherein R₆ and R₈ are independently different or identical and are is selected from H, alkyl, Me, Et, propyl, i-propyl, butyl, iso-butyl, t-butyl, acetyl, acyl, propionyl, benzoyl, or formula (II), or formula (VIII),

R₁₁ and R₁₂ are independently different or identical and are selected from H, Me, Et, propyl, i-propyl, butyl, iso-butyl, t-butyl, acetyl, propionyl, benzoyl, —CH₂Xalkyl, —CH₂Xacyl, where X is O, NR₆ or S,

R_{c} and R_{d} are independently different or identical and are selected from CH₂Xalkyl, CH₂Xacyl, where X═O, NR₆ or S,

R₁₃, R₁₄ and R₁₅ are independently different or identical and are selected from H, Me, Et, propyl, i-propyl, butyl, iso-butyl, t-butyl, —COOH, O-acyl, O-alkyl, N-acyl, N-alkyl, Xacyl, CH₂Xalkyl;

Substituents on R₁₃ and R₁₄ or R₁₃ and R₁₅ may bridge to form a cyclic system to form cycloalkyl, heterocycloalkyl, lactone or lactams.

R_{f}, R_{g}, and Rₕ are independently different or identical and are selected from Xacyl, —CH₂Xalkyl, —CH₂X-acyl and Rg,

alkyl is selected from Me, Et, propyl, i-propyl, butyl, iso-butyl, t-butyl,

acyl is selected from formyl, acetyl, propionyl, isopropionyl, buturyl, tert-butyryl, pentanoyl, benzoyl, succinyl,

acyl and/or alkyl may be optionally substituted, and

when the dotted bond between A and B is present, the compound according to formula (I) is

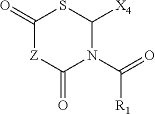

wherein X4 is selected from —COOH, —C(═O)XR₆,

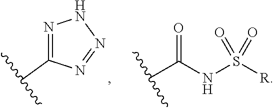

A compound of formula (I), or a pharmaceutically acceptable salt thereof, wherein the dotted bond between A and B denotes an optional bond so as to form a ring closed structure,

and wherein

Z is selected from —CH₂—CH₂— or >CH(CH₃),

A is selected from —SR, —OR and NHR, and R is

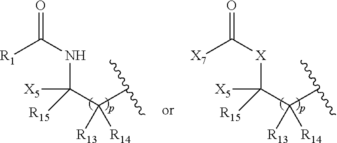

B is selected from —O—R′, —NHR″, —SR′″ or —OH; and R′ is selected from the formula (II) to (IX) below:

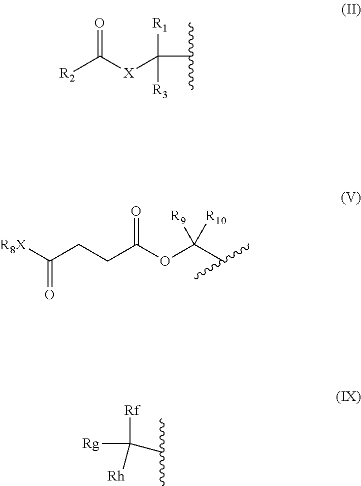

R′, R″ and R′″ are independently different or identical and is selected from formula (IV-VIII) below:

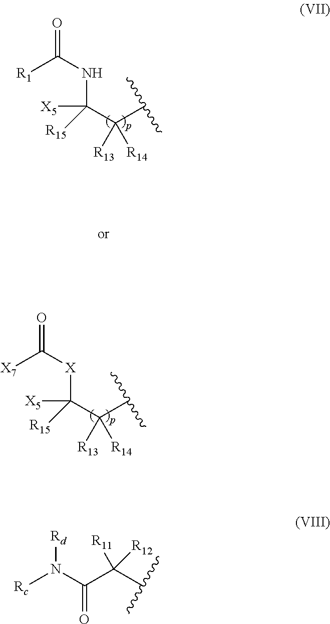

R₁ and R₃ are independently different or identical and are selected from H, Me, Et, propyl, i-propyl, butyl, iso-butyl, t-butyl, O-acyl, O-alkyl, N-acyl, N-alkyl, Xacyl, CH₂Xalkyl, CH₂X-acyl, F, CH₂COOH, CH₂CO₂alkyl,

X is selected from O, NH, NR₆, S,

R₂ is selected from Me, Et, propyl, i-propyl, butyl, iso-butyl, t-butyl, C(O)CH₃,

C(O)CH₂C(O)CH₃, C(O)CH₂CH(OH)CH₃,

p is an integer and is 1 or 2

R₆ is selected from H, Me, Et, propyl, i-propyl, butyl, iso-butyl, t-butyl, acetyl, acyl, propionyl, benzoyl, or formula (II), or formula (VIII)

X₅ is selected from —H, Me, Et, propyl, i-propyl, butyl, iso-butyl, t-butyl, —COOH, —C(═O)XR₆, CONR₁R₃ or is formula

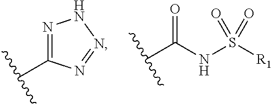

X7 is selected from R₁, —NR₁R₃,

R9 is selected from H, Me, Et or O₂CCH₂CH₂COXR₈

Rio is selected from Oacyl, NHalkyl, NHacyl, or O₂CCH₂CH₂COX₆R₈

X₆ is selected from O, NR₈, NR₆R₈, wherein R₆ and R₈ are independently different or identical and are is selected from H, alkyl, Me, Et, propyl, i-propyl, butyl, iso-butyl, t-butyl, acetyl, acyl, propionyl, benzoyl, or formula (II), or formula (VIII),

R₁₁ and R₁₂ are independently different or identical and are selected from H, Me, Et, propyl, i-propyl, butyl, iso-butyl, t-butyl, acetyl, propionyl, benzoyl, —CH₂Xalkyl, —CH₂Xacyl, where X is O, NR₆ or S,

R_{c} and R_{d} are independently different or identical and are selected from CH₂Xalkyl, CH₂Xacyl, where X═O, NR₆ or S,

R₁₃, R₁₄ and R₁₅ are independently different or identical and are selected from H, Me, Et, propyl, i-propyl, butyl, iso-butyl, t-butyl, —COOH, O-acyl, O-alkyl, N-acyl, N-alkyl, Xacyl, CH₂Xalkyl;

substituents on R₁₃ and R₁₄ or R₁₃ and R₁₅ may bridge to form a cyclic system to form cycloalkyl, heterocycloalkyl, lactone or lactams.

R_{f}, R_{g}, and Rₕ are independently different or identical and are selected from Xacyl, —CH₂Xalkyl, —CH₂X-acyl and Rg,

alkyl is selected from Me, Et, propyl, i-propyl, butyl, iso-butyl, t-butyl,

acyl is selected from formyl, acetyl, propionyl, isopropionyl, buturyl, tert-butyryl, pentanoyl, benzoyl, succinyl,

acyl and/or alkyl may be optionally substituted, and

when the dotted bond between A and B is present, the compound according to formula (I) is

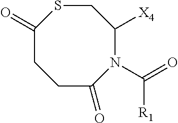

wherein X4 is selected from —COOH, —C(═O)XR₆,

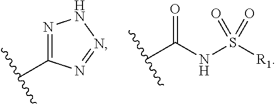

A compound of formula (I), or a pharmaceutically acceptable salt thereof, wherein the dotted bond between A and B denotes an optional bond so as to form a ring closed structure,

and wherein

Z is selected from —CH₂—CH₂— or >CH(CH₃),

A and B are independently different or the same and are selected from —OR, —OR′, —NHR″, —SR′″ or —OH; wherein R is

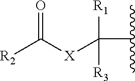

R′ is selected from the formula (II), (V) or (IX) below:

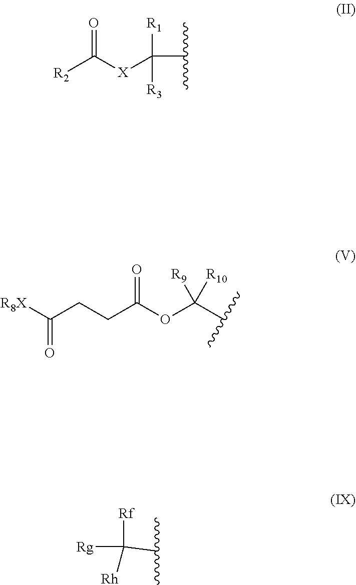

and both A and B are not —OH,

R′, R″ and R′″ are independently different or identical and are selected from formula (VII-VIII) below:

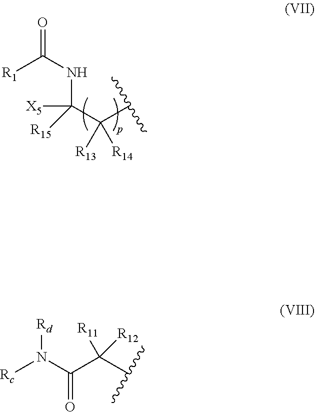

R₁ and R₃ are independently different or identical and are selected from H, Me, Et, propyl, i-propyl, butyl, iso-butyl, t-butyl, O-acyl, O-alkyl, N-acyl, N-alkyl, Xacyl, CH₂Xalkyl, CH₂CH₂CH₂OC(—O)CH₂CH₂COX₆R₈ or

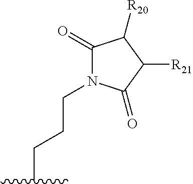

X is selected from O, NH, NR₆, S,

R₂ is selected from Me, Et, propyl, i-propyl, butyl, iso-butyl, t-butyl, C(O)CH₃, C(O)CH₂C(O)CH₃, C(O)CH₂CH(OH)CH₃,

p is an integer and is 1 or 2,

R₆ is selected from H, alkyl, Me, Et, propyl, i-propyl, butyl, iso-butyl, t-butyl, acetyl, acyl, propionyl, benzoyl, or formula (II), or formula (VIII)

X₅ is selected from —H, —COOH, —C(═O)XR₆, CONR₁R₃ or one of the formulas

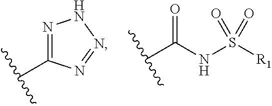

R9 is selected from H, Me, Et or O₂CCH₂CH₂COXR₈,

R10 is selected from Oacyl, NHalkyl, NHacyl, or O₂CCH₂CH₂CO X₆R₈,

X₆ is O or NR₈, and R₈ is selected from H, alkyl, Me, Et, propyl, i-propyl, butyl, iso-butyl, t-butyl, acetyl, acyl, propionyl, benzoyl, succinyl, or formula (II), or formula (VIII),

R₁₁ and R₁₂ are independently the same or different and are selected from H, alkyl, Me, Et, propyl, i-propyl, butyl, iso-butyl, t-butyl, acetyl, acyl, propionyl, benzoyl, succinyl, acyl, —CH₂Xalkyl, —CH₂Xacyl, where X is selected from O, NR₆ or S,

Ris, RM and RI5 are independently different or identical and are selected from H, Me, Et, propyl, i-propyl, butyl, iso-butyl, t-butyl, —COOH, O-acyl, O-alkyl, N-acyl, N-alkyl, Xacyl, CH₂Xalkyl

R_{c} and R_{d} are independently CH₂Xalkyl, CH₂Xacyl, where X═O, NR₆ or S, and alkyl is e.g. H, Me, Et, propyl, i-propyl, butyl, iso-butyl, t-butyl, and acyl is e.g. formyl, acetyl, propionyl, isopropionyl, byturyl, tert-butyryl, pentanoyl, benzoyl, succinyl, or the like,

R_{f}, R_{g}, and Rₕ are independently selected from Xacyl, —CH₂Xalkyl, —CH₂X-acyl and R9, alkyl is selected from methyl, ethyl, propyl, isopropyl, n-butyl, isobutyl, sec-butyl, tert-butyl, n pentyl, neopentyl, isopentyl, hexyl, isohexyl, heptyl, octyl, nonyl or decyl, and acyl is selected from formyl, acetyl, propionyl, butyryl pentanoyl, benzoyl, succinyl and the like,

R₂₀ and R₂₁ are independently different or identical and are selected from H, lower alkyl, i.e. C₁-C₄ alkyl or R₂₀ and R₂₁ together may form a C₄-C₇ cycloalkyl or an aromatic group, both of which may optionally be substituted with halogen, hydroxyl or a lower alkyl, or

R₂₀ and R₂₁ may be

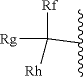

or

CH₂X-acyl, F, CH₂COOH, CH₂CO₂alkyl, and

when there is a cyclic bond present between A and B the compound is

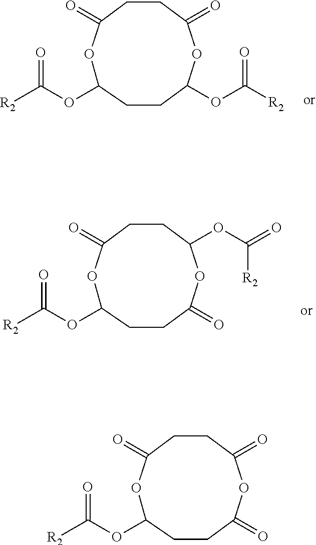

and acyls and alkyls may be optionally substituted.

A compound of formula (A),

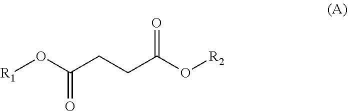

wherein and R₂ are same or different and selected from formula (B)

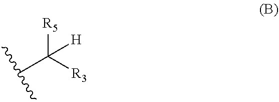

and wherein R₃ is selected from H, or optionally substituted C₁-C₃ alkyl such as e.g., methyl, ethyl, propyl or iso-propyl and wherein R₅ is —OC(═O)Rₐ, wherein Rₐ is methyl or formula (C)

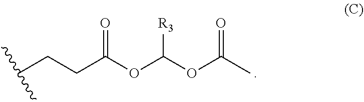

A compound of formula (XX),

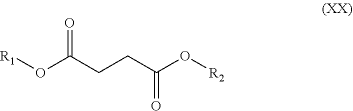

wherein R-\ is H or a pharmaceutically acceptable salt, or an optionally substituted alkyl group or a group of formula (II) and R₂ is independently a group according to formula (II) where formula (II) is

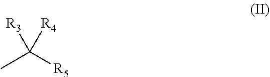

wherein R₃ and R₄ are independently H, optionally substituted C₁-C₃ alkyl, or are linked together to form a ring and where R₅ is linked to R₁ to form a ring or R₅ is selected from OCORₐ, OCOOR_{b}, OCONR_{c}R_{d}, SO₂Rₑ, OPO(OR_{f})(OR_{g}) or CONR_{c}R_{d} where Rₐ is optionally substituted alkyl or optionally substituted cycloalkyl, R_{b} is optionally substituted alkyl, R_{c} and R_{d} are independently H, optionally substituted alkyl or are linked together to form a ring which may contain one or more further heteroatoms, Rₑ is optionally substituted alkyl, R_{f} and R_{g} are independently, H, methyl, ethyl or are linked together to form a ring and wherein the compound is not succinic acid bis (2,2-dimethylpropionyloxymethyl) ester; succinic acid dibutyryloxymethyl ester; or succinic acid bis-(1-butyryloxy-ethyl) ester.

The invention also provides therapeutic compositions that contain citrate, citric acid, or a prodrug, analog, or derivative of citrate or citric acid as one component of combination therapies that also include a composition containing a TCA cycle intermediate or prodrug, analog, or derivative thereof. The compositions that contain citrate, citric acid, or a prodrug, analog, or derivative of citrate or citric acid may be formulated for oral administration, or they may be formulated for non-oral administration.

The compositions of the invention may contain a buffering agent. Any suitable buffering agent may be used. The buffering agent may be an amino acid or a derivative thereof. For example, the amino acid may be alanine, arginine, asparagine, aspartic acid, cysteine, cystine, glutamic Acid, glutamine, glycine, histidine, isoleucine, leucine, lysine, methionine, ornithine, phenylalanine, proline, serine, threonine, tryptophan, tyrosine, or valine. Preferably, the amino acid is lysine or ornithine. The buffering agent may be or may contain a metal ion. For example, the metal ion may be Na⁺, K⁺, Ca²⁺, Me⁺, or Cu²⁺.

The buffering agent may maintain the pH of the composition at neutral or close to neutral. For example and without limitation, the buffering agent may maintain the pH of the composition at from about 3.0 to about 10.0, from about 3.0 to about 9.0, from about 3.0 to about 8.0 from about 3.0 to about 7.0, from about 3.0 to about 6.0, from about 4.0 to about 10.0, from about 4.0 to about 9.0, from about 4.0 to about 8.0 from about 4.0 to about 7.0, from about 4.0 to about 6.0, from about 5.0 to about 10.0, from about 5.0 to about 9.0, from about 5.0 to about 8.0 from about 5.0 to about 7.0, from about 6.0 to about 10.0, from about 6.0 to about 9.0, from about 6.0 to about 8.0 from about 7.0 to about 10.0, from about 7.0 to about 9.0, or from about 8.0 to about 10.0.

In another aspect, the invention provides a formulation including citric acid, or a prodrug, analog, derivative thereof; at least one citrate salt, or a prodrug, analog, or derivative thereof; and an amino acid. The formulation may include citric acid and at least one citrate salt, for example, the at least one citrate salt may be selected from the group consisting of monosodium citrate and monopotassium citrate. In other embodiments, the formulation may be citric acid and at least two citrate salts, for example, the at least two citrate salts may be monosodium citrate and monopotassium citrate.

The formulation may include any natural or non-natural amino acid. In certain embodiments, the amino acid is a cationic amino acid, such as lysine or arginine. The formulation may include other components, such as a sugar, for example sucrose.

The formulation may be provided by any route of administration, and a preferable route is oral. An exemplary oral formulation is a powder that is soluble in an aqueous medium. In a particular embodiment, the formulation includes citric acid, monosodium citrate, monopotassium citrate, sucrose, and one or more of lysine and arginine.

Other aspects of the invention provide methods of treating a condition associated with altered TCA cycle metabolism in a subject. Such methods may involve providing to a subject having a condition associated with altered TCA cycle metabolism a formulation comprising (i) citric acid, or a prodrug, analog, derivative thereof; (ii) at least one citrate salt, or a prodrug, analog, or derivative thereof; and (iii) an amino acid, wherein a therapeutic effect is achieved in the subject by activity of a combination of two or more of components (i), (ii), and (iii) in the formulation. In certain embodiments, the amino acid is incorporated into the TCA cycle of the subject and contributes to the therapeutic effect that is achieved in the subject.

Exemplary conditions include a disorder related to POLG mutation, an energetic disorder, glutaric acidemia type 1 or type 2, a long chain fatty acid oxidation disorder, methylmalonic acidemia (MMA), a mitochondrial associated disease, a mitochondrial encephalomyopathy lactic acidosis and stroke-like syndrome (MELAS), myoclonic epilepsy and ragged-red fibers (MERRF), mitochondrial myopathy, a mitochondrial respiratory chain deficiency, muscular dystrophy (e.g., Duchenne's muscular dystrophy and Becker's muscular dystrophy), a neurologic disorder, a pain or fatigue disease, propionic acidemia (PA), pyruvate carboxylase deficiency, refractory epilepsy, or succinyl CoA lyase deficiency.

The formulation may include citric acid and at least one citrate salt, for example, the at least one citrate salt may be selected from the group consisting of monosodium citrate and monopotassium citrate. In other embodiments, the formulation may be citric acid and at least two citrate salts, for example, the at least two citrate salts may be monosodium citrate and monopotassium citrate.

The formulation may include any natural or non-natural amino acid. In certain embodiments, the amino acid is a cationic amino acid, such as lysine or arginine. The formulation may include other components, such as a sugar, for example sucrose.

The formulation may be provided by any route of administration, and a preferable route is oral. An exemplary oral formulation is a powder that is soluble in an aqueous medium. In a particular embodiment, the formulation includes citric acid, monosodium citrate, monopotassium citrate, sucrose, and one or more of lysine and arginine. The formulation may be provided in one or multiple doses per day. For example, one or more of the formulations may be provided in 1, 2, 3, 4, 5, 6, or more doses per day.

The formulation containing citrate or a prodrug, analog, or derivative thereof may be provided at any therapeutically effective dose. For example, citrate or a prodrug, analog, or derivative thereof may be provided at from about 0.1 mg/kg subject weight to about 5 g/kg subject weight, from about 0.1 mg/kg subject weight to about 5 g/kg subject weight, from about 0.2 mg/kg subject weight to about 2 g/kg subject weight, from about 0.5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

The formulation, or the soluble components within the formulation, may contain citric acid, monosodium citrate, monopotassium citrate, lysine, and sucrose. The formulation may contain, by mass, from about 40% to about 60% citric acid; from about 1% to about 10% monosodium citrate; from about 0.1% to about 5% monopotassium citrate; from about 30% to about 40% lysine; and from about 10% to about 15% sucrose. The formulation, or the soluble components within the formulation, may contain, by mass, about 49.2% citric acid, about 2% monosodium citrate, about 0.3% monopotassium citrate, about 37.5% lysine, and about 11% sucrose. The formulation, or the soluble components within the formulation, may contain, by mass, about 44.2% citric acid, about 8.3% monosodium citrate, about 2.3% monopotassium citrate, about 33.8% lysine, and about 11.5% sucrose. The formulation, or the soluble components within the formulation, may contain, by mass, from about 50% to about 60% citrate, citric acid, or a combination thereof; from about 0.2% to about 1% sodium; from about 0.02% to about 0.5% potassium; from about 30% to about 40% lysine; and from about 10% to about 15% sucrose.

Formulations of the invention may contain aqueous suspensions of one or more TCA cycle intermediates or prodrugs, analogs, or derivatives thereof. The aqueous suspensions may contain the TCA cycle intermediate in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the TCA cycle intermediate in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the TCA cycle intermediate in admixture with a dispersing or wetting agent, suspending agent and one or more preservatives. Suitable dispersing or wetting agents and suspending agents are exemplified, for example sweetening, flavoring and coloring agents, may also be present.

The compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Compositions of the invention may include other pharmaceutically acceptable carriers, such as sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin (glycerol), erythritol, xylitol, sorbitol, mannitol and polyethylene glycol; esters, such asethyl oleate and ethyllaurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Compositions containing TCA cycle intermediates may be in a form suitable for oral use. For example, oral formulations may include tablets, troches, lozenges, fast-melts, aqueous or oily suspensions, dispersible powders or granules, emulsions, hard or soft capsules, syrups or elixirs. Compositions intended for oral use may be prepared according to any method known in the art for the manufacture of pharmaceutical compositions and such compositions may contain one or more agents selected from sweetening agents, flavoring agents, coloring agents and preserving agents, in order to provide pharmaceutically elegant and palatable preparations. Tablets contain citrate or citric acid in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid or talc. The tablets may be uncoated or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108, 4,166,452 and 4,265,874, to form osmotic therapeutic tablets for control release.

Formulations for oral use may also be presented as hard gelatin capsules in which the citrate, citric acid, or a prodrug, analog, or derivative of citrate or citric acid is mixed with an inert solid diluent, for example calcium carbonate, calcium phosphate or kaolin, or as soft gelatin capsules in which the compound is mixed with water or an oil medium, for example peanut oil, liquid paraffin or olive oil.

An alternative oral formulation, where control of gastrointestinal tract hydrolysis of the citrate, citric acid, or a prodrug, analog, or derivative of citrate or citric acid is sought, can be achieved using a controlled-release formulation, where a compound of the invention is encapsulated in an enteric coating.

Syrups and elixirs for oral administration of citrate, citric acid, or a prodrug, analog, or derivative of citrate or citric acid may be formulated with sweetening agents, such as glycerol, propylene glycol, sorbitol or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

The methods of the invention include compositions that are provided as intraocular formulations. Intraocular formulations include any formulation suitable for delivery of an agent to the eye. For example and without limitation, intraocular formulations include aqueous gels, contact lenses, dendrimers, emulsions, emulsions, eye drops, implants, in situ thermosensitive gels, liposomes, microneedles, nanomicelles, nanoparticles, nanosuspensions, ointments, and suspensions. Ocular formulations are known in the art and describe in, for example, Patel, A., et al., Ocular drug delivery systems: An overview, World J Pharmacol. 2013; 2(2): 47-64. doi:10.5497/wjp.v2.i2.47; U.S. Pat. No. 9,636,347; U.S. Publication Nos. 2017/0044274 and 2009/0148527; and International Publication No. WO 2015/105458, the contents of each of which are incorporated herein by reference.

The invention also provides therapeutic compositions that contain the following components: a non-salt form of citrate, citric acid, or a prodrug, analog, or derivative of citrate or citric acid; a nutritional source for infants, such as baby formula or human breast milk; and a buffering agent. The compositions are suitable for oral administration to human infants and provide both basic nutrition and a supplemental source of citrate or citric acid.

The nutritional source may contain one or more of a fat source, a carbohydrate source, and a source of protein or amino acids. The protein source may contain whey and/or casein. The protein source may contain lactose. The fat source may contain a vegetable oil. The nutritional source may contain vitamins and/or minerals.

The nutritional source may be engineered to fulfill the dietary needs of an infant with a metabolic disorder, such as PA or MMA. For example, the source of protein or amino acids may be substantially free of valine, isoleucine, threonine, and methionine. The fat source may be free of odd-chain fatty acids. The nutritional source may be supplemented with carnitine or antibiotics. The nutritional source may have a reduced content of protein and/or amino acids compared to a recommended value for a healthy infant, or it may be substantially free of protein and/or amino acids.

The buffering agent in the compositions containing a non-salt form of citrate or citric acid and a nutritional source may contain any buffering agent, such as those described above. Preferably, the buffering agent is an amino acid, such as lysine or ornithine. The buffering agent may be or may contain a metal ion.

The compositions of the invention may contain one or more antibiotics.

The invention also provides formulations that allow oral delivery of citrate in sufficient quantities to remedy diseases, disorders, and conditions associated with altered TCA cycle metabolism without providing an excessive amount of any mineral or metal ion. The formulations contain amounts of citrate salts such that when the formulation is administered to a person, the cationic component of each salt provided to the person does not exceed the recommended daily amount of that component. In preferred embodiments, the formulation contains each citrate salt in an amount that does not exceed the recommended daily amount of the cation in that citrate salt. Alternatively or additionally, the formulation may comprise a formulation in which the amount of each citrate salt released into the subject's body does not exceed the recommended daily amount of the cation in that citrate salt.

Preferably, the formulation contains the following components: citric acid, or a prodrug, analog, or derivative thereof; one or more citrate salts, or prodrugs, analogs, derivatives thereof; and an amino acid (as an active agent, a buffering agent or both).

The formulation may be a powder that is soluble in an aqueous medium. For example, the formulation may be provided as a dry powder to which water can be added to provide an aqueous solution or suspension that can be consumed orally by a subject. Alternatively, the formulation may be provided as an aqueous solution. The formulation may be provided in a single-serving or a multiple-serving format.

The amino acid may be any amino acid, such as any of those described above. Preferably, the amino acid is lysine, arginine, or ornithine.

Any suitable citrate salt or combination of citrate salts may be used in the formulation. The salt may contain sodium, lithium, potassium, calcium, magnesium, or manganese. The salt may contain citrate in its monobasic, dibasic, or tribasic state. Preferably, the salt is monosodium citrate or monopotassium citrate.

Preferred formulations include citric acid and a combination of citrate salts, such as a combination monosodium citrate and monopotassium citrate. Such combinations allow delivery of ample citrate, in various ionization states, without providing an excess of any individual metal ion.

The recommended daily amount of a salt or mineral may be any amount determined to be suitable for intake for a person in one day. The recommended daily amount may be a minimum amount that should be taken in, a maximum amount that should not be exceeded, or a value or range of values between the two. The recommended daily amount may correspond to a standard known in the art, such as a standard promulgated by a private, governmental, medical, or health agency. For example and without limitation, the standard may be the acceptable daily intake (ADI), issued by the Food and Agriculture Organization and the World Health Organization; the daily intake guide (DIG), issued by the Australian Food and Grocery Council; the daily value (DV) and reference daily intake (RDI), issued by Health Canada; the daily value (DV) or reference daily intake (RDI), issued by the Food and Drug Administration (FDA) in the United States; the dietary reference intake (DRI), estimated average requirement (EAR), or recommended dietary/daily allowance (RDA), issued by the Institute of Medicine (IOM) of the National Academies in the United States; the dietary reference value (DRV), lower reference nutrient intake (LRNI), or reference nutrient intake (RNI), issued by the United Kingdom Department of Health; the dietary reference value (DRV), lower reference nutrient intake (LRNI), or reference nutrient intake (RNI), issued by the European Union's European Food Safety Authority; the guideline daily amount (GDA), issued by the Institute of Grocery Distribution; or the adequate intake (AI), issued by the National Institutes of Health in the United States.

The recommended daily amount may account for conditions related to the individual, such as age, sex, weight, pregnancy status, lactation status, or menopausal status. For example, the recommended daily amounts of several minerals, such as calcium, iron, and potassium, are higher for women who are pregnant and/or lactating than for non-pregnant, non-lactating women. Other groups of patients whose mineral needs may vary include children, (i.e., pediatric patients), post-menopausal women, and the elderly.

For example and without limitation, the recommended daily amounts for specific minerals may be as follows: about 700 mg, about 800 mg, about 1000 mg, about 1200 mg, about 1300 mg, about 1500 mg, about 2000 mg, or about 2500 mg, of calcium; about 0.2 mg, about 0.22 mg, about 0.34 mg, about 0.44 mg, about 0.7 mg, about 0.89 mg, about 0.9 mg, about 1 mg, about 1.3 mg, about 2 mg, about 4 mg, about 6 mg, about 8 mg, or about 10 mg of copper; about 6 mg, about 7 mg, about 8 mg, about 9 mg, about 10 mg, about 11 mg, about 13 mg, about 16 mg, about 18 mg, about 24 mg, about 27 mg, about 30 mg, about 36 mg, about 40 mg, or about 45 mg of iron; about 80 mg, about 150 mg, about 200 mg, about 250 mg, about 310 mg, about 320 mg, about 350 mg, about 360 mg, about 400 mg, or about 420 mg of magnesium; about 1.2 mg, about 1.5 mg, about 1.6 mg, about 1.8 mg, about 1.9 mg, about 2 mg, about 2.2 mg, about 2.3 mg, about 2.6 mg, about 3 mg, about 4 mg, about 6 mg, about 8 mg, about 10 mg, or about 11 mg of manganese; about 3000 mg, about 3500 mg, about 3800 mg, about 4000 mg, about 4500 mg, about 4700 mg, or about 5100 mg of potassium; about 1500 mg, about 2000 mg, about 2300 mg, or about 2400 mg, about 3000 mg, or about 3400 mg of sodium; and about 8 mg, about 9.4 mg, about 11 mg, about 12 mg, about 13 mg, about 15 mg, about 20 mg, about 25 mg, about 30 mg, about 35 mg, or about 40 mg of zinc. The formulation may contain one or more sugars to improve the flavor when the formulation is provided orally to a subject. For example and without limitation, the sugar may be sucrose, fructose, galactose, maltose, or lactose.

The formulation, or the soluble components within the formulation, may contain citric acid, monosodium citrate, monopotassium citrate, lysine, and sucrose. The formulation may contain, by mass, from about 40% to about 60% citric acid; from about 1% to about 10% monosodium citrate; from about 0.1% to about 5% monopotassium citrate; from about 30% to about 40% lysine; and from about 10% to about 15% sucrose. The formulation, or the soluble components within the formulation, may contain, by mass, about 49.2% citric acid, about 2% monosodium citrate, about 0.3% monopotassium citrate, about 37.5% lysine, and about 11% sucrose. The formulation, or the soluble components within the formulation, may contain, by mass, about 44.2% citric acid, about 8.3% monosodium citrate, about 2.3% monopotassium citrate, about 33.8% lysine, and about 11.5% sucrose. The formulation, or the soluble components within the formulation, may contain, by mass, from about 50% to about 60% citrate, citric acid, or a combination thereof; from about 0.2% to about 1% sodium; from about 0.02% to about 0.5% potassium; from about 30% to about 40% lysine; and from about 10% to about 15% sucrose.

### Methods of Treating Metabolic Disorders

The invention provides methods of treating metabolic disorders, diseases, or conditions by providing one or more TCA cycle intermediates or prodrugs, analogs, or derivatives thereof formulated for non-oral administration. The compositions may be administered by any non-oral route. For example and without limitation, the composition may be administered subcutaneously, intravenously, intraarterially, intramuscularly, intradermally, or rectally. Preferably, the composition is administered subcutaneously or intravenously.

The composition containing a TCA cycle intermediate or prodrug, analog, or derivative thereof formulated for non-oral administration may be any of the one described above, such as citrate, cis-aconitate, D-isocitrate, α-ketoglutarate, succinate, fumarate, malate, oxaloacetate, acetone, acetoacetate, β-hydroxybutyrate, β-ketopentanoate, or β-hydroxypentanoate. Preferably, the TCA cycle intermediate is succinate.

The metabolic disorder, disease, or condition may be any disease, disorder, or condition associated with altered TCA cycle metabolism or that can be ameliorated by providing an intermediate of the TCA cycle, such as any of those described above.

The TCA cycle intermediate or prodrug, analog, or derivative thereof may be provided at any therapeutically effective dose. For example and without limitation, the TCA cycle intermediate or prodrug, analog, or derivative thereof may be provided at from about 0.1 mg/kg subject weight to about 5 g/kg subject weight, from about 0.2 mg/kg subject weight to about 5 g/kg subject weight, from about 0.5 mg/kg subject weight to about 5 g/kg subject weight, from about 1 mg/kg subject weight to about 5 g/kg subject weight, from about 2 mg/kg subject weight to about 5 g/kg subject weight, from about 5 mg/kg subject weight to about 5 g/kg subject weight, from about 0.1 mg/kg subject weight to about 2 g/kg subject weight, from about 0.2 mg/kg subject weight to about 2 g/kg subject weight, from about 0.5 mg/kg subject weight to about 2 g/kg subject weight, from about 1 mg/kg subject weight to about 2 g/kg subject weight, from about 2 mg/kg subject weight to about 2 g/kg subject weight, from about 5 mg/kg subject weight to about 2 g/kg subject weight, from about 0.1 mg/kg subject weight to about 1 g/kg subject weight, from about 0.2 mg/kg subject weight to about 1 g/kg subject weight, from about 0.5 mg/kg subject weight to about 1 g/kg subject weight, from about 1 mg/kg subject weight to about 1 g/kg subject weight, from about 2 mg/kg subject weight to about 1 g/kg subject weight, from about 5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

The composition may be provided according to any suitable schedule. For example and without limitation, the composition may be provided as single dose every 1 hour, 2 hours, 4 hours, 6 hours, 8 hours, 12 hours, 1 day, 2 days, 3 days, 4 days, 5 days, or more. Each formulation independently may be provided once, twice, three times, four times, or more per day.

The composition may be provided over a period of time. For example and without limitation, the composition may be provided for 1 day, 2 days, 3 days, 4 days, 5 days, 6 days, 1 week, 2 weeks, 3 weeks, 4 weeks, 6 weeks, 8 weeks, 12 weeks, 3 months, 4 months, 5 months, 6 months, 8 months, 10 months, 12 months, 18 months, or 24 months. One or more boundaries of the period may be defined by the age of the patient. For example and without limitation, the period of providing the composition may start or end when the patient is born, 1 week old, 2 weeks old, 3 weeks old, 4 weeks old, 6 weeks old, 8 weeks old, 10 weeks old, 12 weeks old, 3 months old, 3 months old, 4 months old, 5 months old, 6 months old, 8 months old, 10 months old, 12 months old, 18 months old, or 24 months old.

The invention provides combination therapies for treating metabolic disorders, diseases, or conditions. The combination therapies include providing a non-oral formulation containing succinate or a prodrug, analog, or derivative thereof and another formulation containing citrate, citric acid, or a prodrug, analog, or derivative thereof. The non-oral formulation may be administered by any non-oral route. For example and without limitation, the non-oral formulation may be administered subcutaneously, intravenously, intraarterially, intramuscularly, intradermally, or rectally. Preferably, the non-oral formulation is administered subcutaneously or intravenously. The formulation containing citrate, citric acid, or a prodrug, analog, or derivative of citrate or citric acid may be administered by any route, such as orally, enterally, parenterally, subcutaneously, intravenously, intraarterially, intramuscularly, intradermally, or rectally. Preferably, the formulation containing citrate, citric acid, or a prodrug, analog, or derivative of citrate or citric acid is provided orally.

The combination therapies of the invention may be used to treat any disease, disorder, or condition associated with altered TCA cycle metabolism or that can be ameliorated by providing an intermediate of the TCA cycle, such as any of those described above.

In the combination therapies of the invention, each of succinate or a prodrug, analog, or derivative thereof and citrate or a prodrug, analog, or derivative of citrate or citric acid is provided at therapeutically effective dose. For example, each independently may be provided at from about 0.1 mg/kg subject weight to about 5 g/kg subject weight, from about 0.1 mg/kg subject weight to about 5 g/kg subject weight, from about 0.2 mg/kg subject weight to about 2 g/kg subject weight, from about 0.5 mg/kg subject weight to about 1 g/kg subject weight, from about 1 mg/kg subject weight to about 500 mg/kg subject weight, from about 2 mg/kg subject weight to about 200 mg/kg subject weight, or from about 5 mg/kg subject weight to about 100 mg/kg subject weight.

In the combination therapies of the invention, the non-oral formulation containing succinate or a prodrug, analog, or derivative thereof and the other formulation containing citrate, citric acid, or a prodrug, analog, or derivative of citrate or citric acid may be provided in any temporal relationship. For example, the two formulations may be provided simultaneously, sequentially in either order, or in alternating sequence. In a preferred method, the non-oral formulation containing succinate or a prodrug, analog, or derivative thereof is provided first, and the formulation containing citrate, citric acid, or a prodrug, analog, or derivative of citrate or citric acid is provided subsequently.

In the combination therapies of the invention, each formulation independently may be provided according to any suitable schedule, as described above.

In the combination therapies of the invention, each formulation independently may be provided over a period of time, as described above.

In the combination therapies of the invention, the non-oral formulation containing succinate or a prodrug, analog, or derivative thereof and the other formulation containing citrate, citric acid, or a prodrug, analog, or derivative of citrate or citric acid may be provided over distinct periods, coextensive periods, or overlapping periods. Thus, the combination therapies may include transitions from a period in which one formulation is provided to a period in which the other formulation is provided. The transitions may be discrete, or they may contain periods in which both formulations are provided. The transitions may be gradual and may include a decrease in dosage of the first formulation and an increase in dosage of the second formulation.

### EXAMPLES

### Example 1: Subcutaneous Administration of Succinate

Summary

The bioavailability of ¹³C₄-succinate was determined following subcutaneous (SC) administration in male Sprague-Dawley rats. ¹³C₄-succinate was dosed subcutaneously at 10 mg/kg, 50 mg/kg, and 100 mg/kg. Blood samples were collected up to 8 hours post-dose, and plasma concentrations of the test article were determined by LC-MS/MS. Pharmacokinetic parameters were determined using Phoenix WinNonlin (v8.0) software.

Following SC dosing of ¹³C₄-succinate at 10 mg/kg, maximum plasma concentrations (average of 3480±1512 ng/mL) were observed at 15 minutes post dosing. The average half-life following subcutaneous dosing could not be determined; however, the half-life for one rat was 0.453 hours. The average exposure based on the dose-normalized AUCₗₐₛₜ was 160±55.4 hr*kg*ng/mL/mg. Based on the IV data from a previous study, the average bioavailability of ¹³C₄-succinate at 10 mg/kg was 74.4±25.7%.

Following SC dosing of ¹³C₄-succinate at 50 mg/kg, maximum plasma concentrations (average of 17333±2214 ng/mL) were observed at 15 minutes post dosing. The average half-life following subcutaneous dosing could not be determined because the correlation coefficient (r²) was less than 0.85. The average exposure based on the dose-normalized AUCₗₐₛₜ was 210±41.0 hr*kg*ng/mL/mg. Based on the IV data from a previous study, the average bioavailability of ¹³C₄-succinate at 50 mg/kg was 97.6±19.0%.

Following SC dosing of ¹³C₄-succinate at 100 mg/kg, maximum plasma concentrations (average of 31000±7451 ng/mL) were observed at 15 minutes post dosing. The average half-life following subcutaneous dosing was 1.07 hours. The average exposure based on the dose-normalized AUCₗₐₛₜ was 216±52.9 hr*kg*ng/mL/mg. Based on the IV data from a previous study, the average bioavailability of ¹³C₄-succinate at 100 mg/kg was 100±24.6%.

Following subcutaneous dosing of ¹³C₄-succinate at 10, 50, and 100 mg/kg, there was a nonlinear increase in exposure with increasing dose. The exposure of the higher doses was dose proportional, but greater than dose proportional to the low dose exposure.

Observations and Adverse Reactions

No adverse reactions were observed following subcutaneous dosing of ¹³C₄-succinate in male Sprague-Dawley rats.

Dosing Solution Analysis

The dosing solutions were analyzed by LC-MS/MS. The measured dosing solution concentrations are shown in Table 1. The dosing solutions were diluted into rat plasma and analyzed in triplicate. All concentrations are expressed as mg/mL of the free base. The nominal dosing level was used in all calculations.

TABLE 1
Measured Dosing Solution Concentrations (mg/mL)

| | | | Nominal | Measured | |
|---|---|---|---|---|---|
| | Route of | | Dosing | Dosing | |
| Test | Adminis- | | Conc. | Conc. | % of |
| Article | tration | Vehicle | (mg/mL) | (mg/mL) | Nominal |
| ¹³C₄- | SC | Physiologic | 2 | 2.14 | 107 |
| succinate | SC | Saline | 10 | 9.75 | 97.5 |
| | SC | buffered to | 20 | 19.5 | 97.3 |
| | | pH 6.5-7 | | | |

Quantitative Plasma Sample Analysis

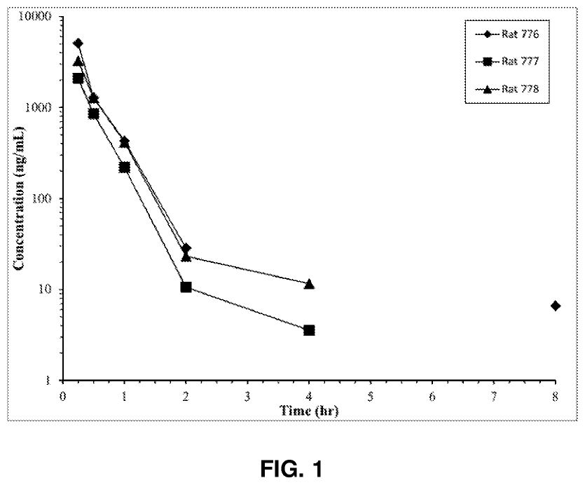

Plasma samples were extracted and analyzed using the methods described below in the section on Sample Extraction. Individual and average plasma concentrations are shown in Table 2 through Table 4. All data are expressed as ng/mL of the free base. Samples that were below the limit of quantification were not used in the calculation of averages. Concentrations versus time data are plotted in FIG. 1 through FIG. 6.

Data Analysis

Pharmacokinetic parameters were calculated from the time course of the plasma concentration and are presented in Table 2 through Table 4. Pharmacokinetic parameters were determined with Phoenix WinNonlin (v8.0) software using a non-compartmental model. The maximum plasma concentration (Cₘₐₓ) and the time to reach maximum plasma concentration (tₘₐₓ) after SC dosing were observed from the data. The area under the time-concentration curve (AUC) was calculated using the linear trapezoidal rule with calculation to the last quantifiable data point, and with extrapolation to infinity if applicable. Plasma half-life (t_{1/2}) was calculated from 0.693/slope of the terminal elimination phase. Mean residence time, MRT, was calculated by dividing the area under the moment curve (AUMC) by the AUC. Bioavailability was determined by dividing the individual dose-normalized PO AUCₗₐₛₜ values by the average dose-normalized IV AUCₗₐₛₜ value from study 18CARNP11. Any samples below the limit of quantitation (1.00 ng/mL) were treated as zero for pharmacokinetic data analysis.

TABLE 2
Individual and Average Plasma Concentrations (ng/mL) for ¹³C₄-Succinate
After Subcutaneous Administration at 10 mg/kg in Male Sprague-Dawley Rats
Subcutaneuus (10 mg/kg)

| | Rat # | | | | |
|---|---|---|---|---|---|
| Time (hr) | 776 | 777 | 778 | Mean | SD |
| 0 (pre-dose) | BLOQ | BLOQ | BLOQ | ND | ND |
| 0.25 | 5090 | 2090 | 3260 | 3480 | 1512 |
| 0.50 | 1280 | 854 | 1280 | 1138 | 246 |
| 1.0 | 430 | 220 | 415 | 355 | 117 |
| 2.0 | 28.5 | 10.6 | 23.2 | 20.8 | 9.19 |
| 4.0 | BLOQ | 3.57 | 11.6 | 7.59 | ND |
| 8.0 | 6.63 | BLOQ | BLOQ | ND | ND |
| Animal Weight (kg) | 0.263 | 0.262 | 0.275 | 0.267 | 0.007 |
| Volume Dosed (mL) | 1.32 | 1.31 | 1.38 | 1.34 | 0.04 |
| Cₘₐₓ (ng/mL) | 5090 | 2090 | 3260 | 3480 | 1512 |
| tₘₐₓ (hr) | 0.25 | 0.25 | 0.25 | 0.25 | 0.00 |
| t_{1/2} (hr) | ND³ | 0.453 | ND³ | ND | ND |
| MRTₗₐₛₜ (hr) | 0.503 | 0.489 | 0.527 | 0.506 | 0.0194 |
| AUCₗₐₛₜ (hr · ng/mL) | 2131 | 1027 | 1653 | 1604 | 554 |
| AUC_{∞} (hr · ng/mL) | ND³ | 1030 | ND³ | ND | ND |
| Dose-normalized | | | | | |
| Values¹ | | | | | |
| AUCₗₐₛₜ | 2.13 | 103 | 165 | 160 | 55.4 |
| (hr · kg · ng/mL/mg) | | | | | |
| AUC_{∞} | ND³ | 103 | ND³ | ND | ND |
| (hr · kg · ng/mL/mg) | | | | | |
| Bioavailability (%)² | 98.9 | 47.7 | 76.7 | 74.4 | 25.7 |

Cₘₐₓ: maximum plasma concentration; tₘₐₓ: time of maximum plasma concentration; t_{1/2}: half-life, data points used for half-life determination are in bold; MRTₗₐₛₜ: mean residence time, calculated to the last observable time point; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Dose-normalized by dividing the parameter by the nominal dose in mg/kg.
²Bioavailability determined by dividing the individual oral dose-normalized AUCₗₐₛₜ values by the average IV dose normalized AUCₗₐₛₜ value 215 hr*kg*ng/mL/mg from previous study.
³Not determined because the line defining the terminal elimination phase had an r² < 0.85.

FIG. 1 is a graph of plasma concentration of ¹³C₄-succinate at various time points in individual rats after subcutaneous administration of ¹³C₄-succinate at 10 mg/kg.

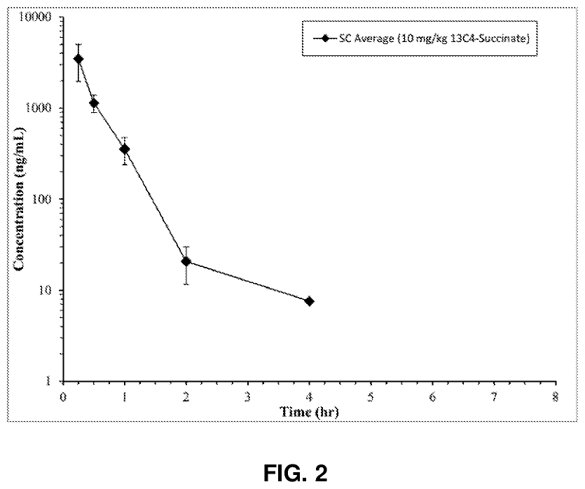

FIG. 2 is a graph of average plasma concentration of ¹³C₄-succinate at various time points in rats after subcutaneous administration of ¹³C₄-succinate at 10 mg/kg.

TABLE 3
Individual and Average Plasma Concentrations (ng/mL) for ¹³C₄-Succinate
After Subcutaneous Administration at 50 mg/kg in Male Sprague-Dawley Rats
Subcutaneous (50 mg/kg)

| | Rat # | | | | |
|---|---|---|---|---|---|
| Time (hr) | 779 | 780 | 781 | Mean | SD |
| 0 (pre-dose) | BLOQ | BLOQ | BLOQ | ND | ND |
| 0.25 | 14800 | 18300 | 18900 | 17333 | 2214 |
| 0.50 | 9750 | 15500 | 11100 | 12117 | 3007 |
| 1.0 | 1020 | 2420 | 2020 | 1820 | 721 |
| 2.0 | 102 | 163 | 122 | 129 | 31.1 |
| 4.0 | 15.1 | 10.3 | 12.3 | 12.6 | 2.41 |
| 8.0 | 42.8 | 8.84 | 4.69 | 18.8 | 20.9 |
| Animal Weight (kg) | 0.275 | 0.265 | 0.255 | 0.265 | 0.010 |
| Volume Dosed (mL) | 1.38 | 1.33 | 1.28 | 1.33 | 0.05 |
| Cₘₐₓ (ng/mL) | 14800 | 18300 | 18900 | 17333 | 2214 |
| tₘₐₓ (hr) | 0.25 | 0.25 | 0.25 | 0.25 | 0.00 |
| t_{1/2} (hr) | ND³ | ND³ | ND³ | ND | ND |
| MRTₗₐₛₜ (hr) | 0.558 | 0.530 | 0.505 | 0.531 | 0.0267 |
| AUCₗₐₛₜ (hr · ng/mL) | 8405 | 12496 | 10632 | 10511 | 2048 |
| AUC_{∞} (hr · ng/mL) | ND³ | ND³ | ND³ | ND | ND |
| Dose-normalized | | | | | |
| Values¹ | | | | | |
| AUCₗₐₛₜ | 168 | 250 | 213 | 210 | 41.0 |
| (hr · kg · ng/mL/mg) | | | | | |
| AUC_{∞} | ND³ | ND³ | ND³ | ND | ND |
| (hr · kg · ng/mL/mg) | | | | | |
| Bioavailability (%)² | 78.0 | 116 | 98.7 | 97.6 | 19.0 |

Cₘₐₓ: maximum plasma concentration; tₘₐₓ: time of maximum plasma concentration; t_{1/2}: half-life, data points used for half-life determination are in bold; MRTₗₐₛₜ: mean residence time, calculated to the last observable time point; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Dose-normalized by dividing the parameter by the nominal dose in mg/kg.
²Bioavailability determined by dividing the individual oral dose-normalized AUCₗₐₛₜ values by the average IV dose normalized AUCₗₐₛₜ value 215 hr*kg*ng/mL/mg from previous study.
³Not determined because the line defining the terminal elimination phase had an r² < 0.85.

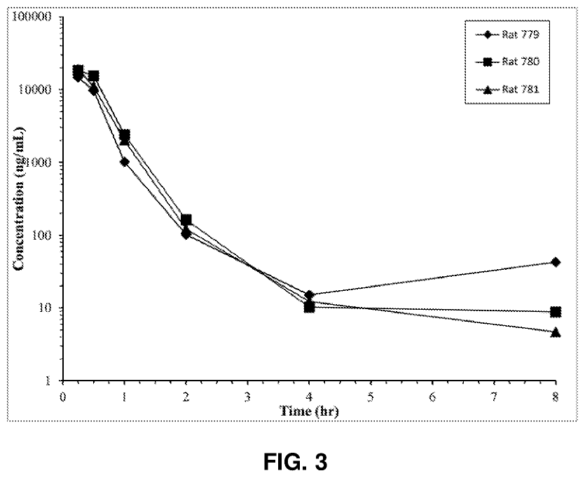

FIG. 3 is a graph of plasma concentration of ¹³C₄-succinate at various time points in individual rats after subcutaneous administration of ¹³C₄-succinate at 50 mg/kg.

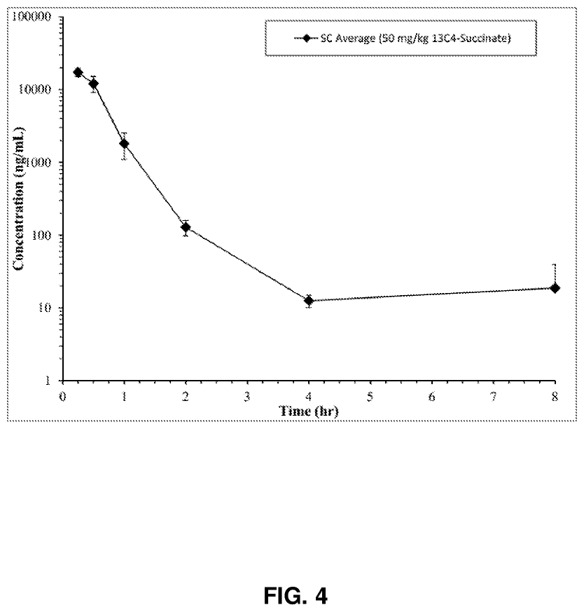

FIG. 4 is a graph of average plasma concentration of ¹³C₄-succinate at various time points in rats after subcutaneous administration of ¹³C₄-succinate at 50 mg/kg.

TABLE 4
Individual and Average Plasma Concentrations (ng/mL) for ¹³C₄-Succinate After
Subcutaneous Administration at 100 mg/kg in Male Sprague-Dawley Rats
Subcutaneous (100 mg/kg)

| | Rat # | | | | |
|---|---|---|---|---|---|
| Time (hr) | 782 | 783 | 784 | Mean | SD |
| 0 (pre-dose) | BLOQ | BLOQ | BLOQ | ND | ND |
| 0.25 | 37900 | 23100 | 32000 | 31000 | 7451 |
| 0.50 | 24500 | 13900 | 25200 | 21200 | 6332 |
| 1.0 | 6840 | 4980 | 7750 | 6523 | 1412 |
| 2.0 | 364 | 435 | 779 | 526 | 222 |
| 4.0 | 103 | 40.8 | 52.9 | 65.6 | 33.0 |
| 8.0 | 17.7 | 10.5 | 49.5 | 25.9 | 20.8 |
| Animal Weight (kg) | 0.257 | 0.258 | 0.271 | 0.262 | 0.008 |
| Volume Dosed (mL) | 1.29 | 1.29 | 1.36 | 1.31 | 0.0 |
| Cₘₐₓ (ng/mL) | 37900 | 23100 | 32000 | 31000 | 7451 |
| tₘₐₓ (hr) | 0.25 | 0.25 | 0.25 | 0.25 | 0.00 |
| t_{1/2} (hr) | 1.40 | 0.747 | ND³ | 1.07 | ND |
| MRTₗₐₛₜ (hr) | 0.596 | 0.628 | 0.660 | 0.628 | 0.0323 |
| AUCₗₐₛₜ (hr · ng/mL) | 24683 | 15518 | 24689 | 21630 | 5293 |
| AUC_{∞} (hr · ng/mL) | 24719 | 15530 | ND³ | 20124 | ND |
| Dose-normalized | | | | | |
| Values¹ | | | | | |
| AUCₗₐₛₜ | 247 | 155 | 247 | 216 | 52.9 |
| (hr · kg · ng/mL/mg) | | | | | |
| AUC_{∞} | 247 | 155 | ND³ | 201 | ND |
| (hr · kg · ng/mL/mg) | | | | | |
| Bioavailability (%)² | 115 | 72.0 | 115 | 100 | 24.6 |

Cₘₐₓ: maximum plasma concentration; tₘₐₓ: time of maximum plasma concentration; t_{1/2}: half-life, data points used for half-life determination are in bold; MRTₗₐₛₜ: mean residence time, calculated to the last observable time point; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Dose-normalized by dividing the parameter by the nominal dose in mg/kg.
²Bioavailability determined by dividing the individual oral dose-normalized AUCₗₐₛₜ values by the average IV dose normalized AUCₗₐₛₜ value 215 hr*kg*ng/mL/mg from previous study.
³Not determined because the line defining the terminal elimination phase had an r² < 0.85.

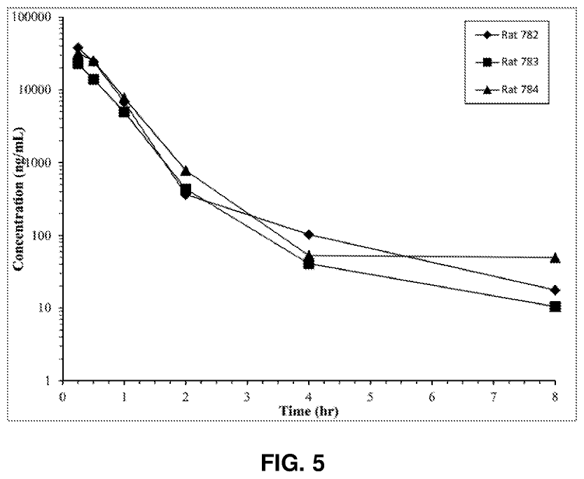

FIG. 5 is a graph of plasma concentration of ¹³C₄-succinate at various time points in individual rats after subcutaneous administration of ¹³C₄-succinate at 100 mg/kg.

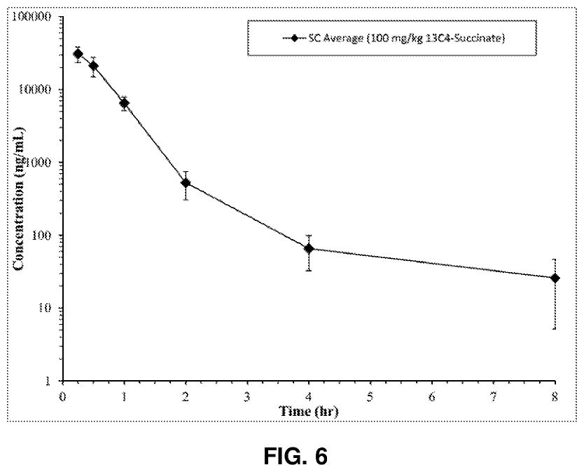

FIG. 6 is a graph of average plasma concentration of ¹³C₄-succinate at various time points in rats after subcutaneous administration of ¹³C₄-succinate at 100 mg/kg.

Standard Preparation

Standards were prepared in Sprague-Dawley rat plasma containing sodium heparin as the anticoagulant. Working solutions were prepared in 50:50 acetonitrile:water and then added to the rat plasma to make calibration standards to final concentrations of 2000, 1000, 500, 100, 50.0, 10.0, 5.00, and 1.00 ng/mL. Standards were treated identically to the study samples

Sample Extraction

Plasma samples were manually extracted using acetonitrile in a 96-well plate as outlined in Table 5.

TABLE 5
Preparation of Plasma Samples

| Step | Procedure |
|---|---|
| 1 | Standards: Add 10 μL of appropriate working solution to 50 μL |
| | of blank plasma. |
| | Blanks: Add 10 μL 50:50 acetonitrile:water to 50 μL of blank |
| | plasma. |
| | Samples: Add 10 μL 50:50 acetonitrile:water to 50 μL of study |
| | sample. |
| | Cap and mix. |
| 2 | Add 150 μL of acetonitrile containing 100 ng/mL of warfarin as |
| | internal standard. |
| | Cap and vortex well. |
| 3 | Centrifuge samples at 3000 rpm for 5 minutes. |
| 4 | Evaporate 100 μL of supernatant and reconstitute with 100 μL |
| | of Milli-Q water. |

### Example 2: Effects of Citrate and Succinate on Basal Respiration

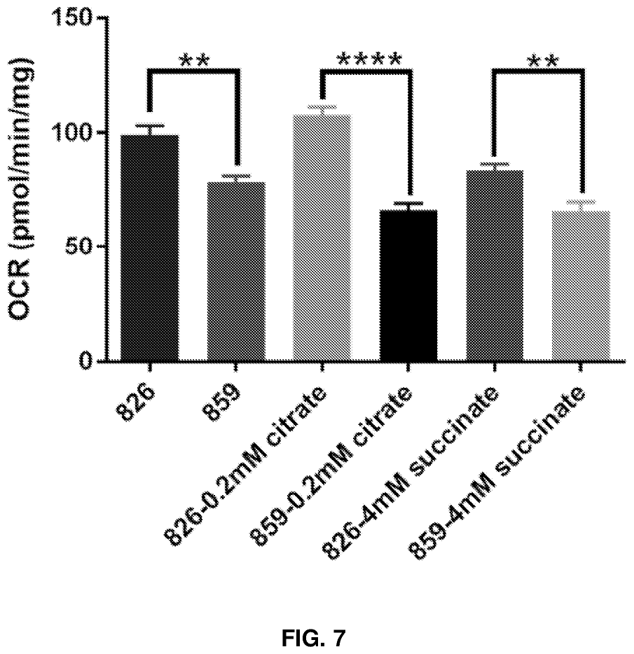

FIG. 7 is graph showing the effects of citrate and succinate on basal respiration in cells from a patient with propionic acidemia (PA). Fibroblasts from a normal patient (sample 826) and fibroblasts from a patient with PA were cultured for 72 hours in culture medium alone, culture medium supplemented with 0.2 mM citrate, or culture medium supplemented with 4 mM succinate, as indicated. Oxygen consumption rate (OCR) was measured with an XFe96 Extracellular Flux Analyzer, Seahorse Bioscience. Data shown are mean values±SD and are normalized to protein amount mean value. ****P<0.0001,**P<0.01 compared between the groups shown are calculated in GraphPad Prism 7 using unpaired t test for samples from a single biological replicate and 8 technical replicates.

### Example 3: Pharmacokinetics, Excretion, and Tissue Distribution of Orally-Administered Citrate or Intravenously-Administered Succinate

Summary

The plasma-time course, routes and rates of excretion, and tissue distribution of radioactivity following a single oral dose of ¹⁴C-labeled citrate or single oral dose of ¹⁴C-labeled succinate were analyzed in rats.

Dose Formulation and Preparation

¹⁴C-succinate and ¹⁴C-citrate were dissolved in either deionized water for oral administration or 0.9% sodium chloride for intravenous injection. Formulations were prepared on the day prior to dosing.

Test System

CD® rats were obtained from Charles River Laboratories. Animals were 9-11 weeks in age at beginning of study.

Study Design

Treatment groups are shown in Table 6.

TABLE 6
Treatment Groups

| | | Dose | Dose | Radioactive | |
|---|---|---|---|---|---|
| | | Level | Volume | Dose | No. of |
| Group | Treatment | (mg/kg) | (mL/kg) | (μCi/kg) | Animals |
| 1 | ¹⁴C-succinate IV | 10 | 1 | 200 | 6 |
| 2 | ¹⁴C-citrate Oral | 100 | 5 | 200 | 6 |

Animals given oral ¹⁴C-citrate were kept in a fasting state for 8 hours prior to dosing and for 4 hours after dosing. ¹⁴C-citrate was given by oral gavage. Animals given intravenous ¹⁴C-succinate were not kept in a fasting state. ¹⁴C-succinate was administered by tail vein injection.

Sample Collection and Analysis

Following oral dosing, the tube and dose site were wiped with gauze, which was collected for subsequent radioanalysis. Following IV dosing, the needle and dose site were wiped with gauze, which was collected for subsequent radioanalysis. The dose site wipe total radioactivity was subtracted from the total dose administered, to determine dose loss, according to SOP ADME.

Blood and plasma were collected by cardiac puncture for terminal collection at 0.5, 1, 2, 4, 8, and 24 hours post-dose. Blood was stored on an ice block or wet ice until centrifugation to obtain plasma. Triplicate aliquots were weighed, mixed with scintillation cocktail, and counted by LSC.

Urine and feces were collected at 0-24 hours post-dose and stored on wet ice. Expired CO₂ was collected at 0-8 hours and 8-24 hours post-dose by drawing air through traps containing 2 N NaOH according to MPI Research SOP. Samples were stored at ambient temperature and weighed. Aliquots were submitted for radioanalysis.

At 24 hours post-dose, cages were rinsed with a 1% trisodium phosphate (TSP) solution and wiped with gauze pads. The final cage rinse samples and cage wipe samples were collected into separate appropriate containers, and the weight of each cage rinse and cage wipe were recorded.

Samples were analyzed for radioactivity by liquid scintillation counting (LSC) for at least 5 minutes or 100,000 cumulative counts. Samples were analyzed in triplicate.

At 0.5, 1, 2, 4, 8, and 24 hours post-dose, after terminal blood collections, euthanized animals were frozen in a hexane/dry ice bath and embedded in a 5% carboxymethylcellulose matrix according to MPI Research SOP IMG-23.

Embedded carcasses were sectioned according to MPI Research SOP EQP-145 on a Leica CM3600 Cyromacrotome set to maintain −10 to −30° C. Sections of approximately 30 μm thickness were taken in the sagittal plane. Appropriate sections selected at various levels of interest in the block were collected to encompass the following tissues, organs, and biological fluids, where possible: adrenal gland, bladder and contents (urine), blood (cardiac), bone, bone marrow, brain, epididymis, eye, fat (brown), fat (white), heart, kidney, large intestine/cecum and contents, liver, lung, muscle, pancreas, prostate/uterus, salivary glands, skin, small intestine and contents, spleen, stomach and contents, testes/ovaries, thymus, thyroid, and mesenteric lymph nodes. Sections were mounted, exposed to phosphor imaging screens, and scanned at 50 μm using the Storm 860 image acquisition system according to MPI Research SOP EQP-146.

Quantification, relative to the calibration standards, was performed by image densitometry using MCID™ image analysis software according to MPI Research SOP IMG-24. A standard curve was constructed from the integrated response (MDC/mm²) and the nominal concentrations of the [¹⁴C]calibration standards. The concentrations of radioactivity were expressed as μCi/g and converted to μg or ng equivalents of [¹⁴C]Test Article per gram sample (ng-eq/g or μg-eq/g) using the specific activity of administered [¹⁴C]Test Article dose formulation. A lower limit of quantification (LLOQ) was applied to the data. The LLOQ was determined using the radioactive concentration of the lowest calibration standard used to generate a calibration curve divided by the specific activity of the dose formulation (μCi/mg). Artifacts, such as those produced by dislodged contents of the alimentary canal, were excluded from analysis during image analysis.

Samples were stored as indicated in Table 7.

TABLE 7
Sample Storage Conditions

| Sample | Storage Condition (range) | Comments |
|---|---|---|
| Blood | Wet ice prior to preparing | Discard cell pellet |
| | plasma | |
| Plasma | Frozen (−10 to −30° C.) | |
| Urine | Frozen (−10 to −30° C.) | |
| Feces | Frozen (−10 to −30° C.) | |
| Cage Rinses | Ambient | Discard following |
| | | analysis |
| Cage Wipes | Ambient | Discard following |
| | | analysis |
| Carcasses | Frozen (−10 to −30° C.) | Carcasses for |
| | | QWBA |
| Dose Site | Ambient | Discard following |
| Wipes | | analysis |
| Expired Air | Ambient | |

Results

The percentages of radioactivity recovered in various samples from animals sacrificed 24 hours after treatment with either a single dose of oral ¹⁴C-citrate or a single dose of intravenous ¹⁴C-succinate are shown in Table 8.

TABLE 8
Percentage Recovery of Radioactive Dose

| | | | ¹⁴C-succinate | ¹⁴C-citrate |
|---|---|---|---|---|
| | | | IV Treatment, | Oral Treatment, |
| | | | Subject 1006 | Subject 2006 |
| Sample | Timepoint | | (% recovery) | (% recovery) |
| Expired trap 1 | 0-8 | hr | 54.8 | 2006 |
| Expired trap 1 | 0-8 | hr | 4.6 | 40.0 |
| Expired trap 1 | 0-8 | hr | 0.4 | 16.4 |
| Subtotal | | | 59.8 | 2.7 |
| Expired trap 1 | 0-8 | hr | 1.3 | 59.1 |
| Expired trap 1 | 0-8 | hr | 0.7 | 1.2 |
| Expired trap 1 | 0-8 | hr | 0.3 | 0.9 |
| Subtotal | | | 2.3 | 0.4 |
| Urine | 0-24 | hr | 2.9 | 5.1 |
| Feces | 0-24 | hr | 0.2 | 0.4 |
| Cage rinse | 0-24 | hr | 0.1 | 0.1 |
| Cage wipe | 24 | hr | 0.0 | 0.0 |

| | | | |
|---|---|---|---|
| Total | Total | 65.265 | 67.285 |

The plasma concentrations of radioactive material in rats treated with either a single dose of oral ¹⁴C-citrate or a single dose of intravenous ¹⁴C-succinate are shown in Table 9.

TABLE 9
Plasma Concentrations of Radioactive Material

| | | | | | Concentration |
|---|---|---|---|---|---|
| Treatment | Subject | Sample | Time | | (ng-equivalents/g) |
| ¹⁴C-succinate IV | 1001 | Plasma | 30 | min | 2809 |
| ¹⁴C-succinate IV | 1002 | Plasma | 1 | hr | 2996 |
| ¹⁴C-succinate IV | 1003 | Plasma | 2 | hr | 1466 |
| ¹⁴C-succinate IV | 1004 | Plasma | 4 | hr | 1158 |
| ¹⁴C-succinate IV | 1005 | Plasma | 8 | hr | 813 |
| ¹⁴C-succinate IV | 1006 | Plasma | 24 | hr | 511 |
| ¹⁴C-citrate Oral | 2001 | Plasma | 30 | min | 32073 |
| ¹⁴C-citrate Oral | 2002 | Plasma | 1 | hr | 31322 |
| ¹⁴C-citrate Oral | 2003 | Plasma | 2 | hr | 26187 |
| ¹⁴C-citrate Oral | 2004 | Plasma | 4 | hr | 21812 |
| ¹⁴C-citrate Oral | 2005 | Plasma | 8 | hr | 16472 |
| ¹⁴C-citrate Oral | 2006 | Plasma | 24 | hr | 6762 |

The concentrations of radioactivity in blood and tissues in rats treated with a single dose of intravenous ¹⁴C-succinate are shown in Table 10.

TABLE 10
Radioactivity in Tissues of Rats Treated with IV ¹⁴C-Succinate

| | ng Equivalents test article/g | | | | | |
|---|---|---|---|---|---|---|
| | Animal Number (Timepoint) | | | | | |
| | 1001 | 1002 | 1003 | 1004 | 1005 | 1006 |
| Tissue | (0.5 Hour) | (1 Hour) | (2 Hours) | (4 Hours) | (8 Hours) | (24 Hours) |
| Adrenal gland | 4060 | 1441 | 1188 | 1192 | 840 | 581 |
| Blood | 891 | 1398 | 912 | BLQ | BLQ | BLQ |
| Bone | 3128 | 3200 | 1989 | 2330 | 2042 | 1322 |
| Bone marrow | 1314 | 1750 | 1398 | 1419 | 1019 | 769 |
| Cecum | BLQ | BLQ | BLQ | BLQ | BLQ | BLQ |
| Cecum contents | 540 | 882 | 757 | 750 | 806 | BLQ |
| Cerebellum | 709 | 847 | 529 | 503 | BLQ | BLQ |
| Cerebrum | 596 | 853 | 477 | 543 | 348 | BLQ |
| Epididymis | 699 | 785 | 442 | BLQ | BLQ | BLQ |
| Eye | 569 | 584 | 371 | 413 | BLQ | BLQ |
| Fat (brown) | 996 | BLQ | BLQ | BLQ | BLQ | BLQ |
| Fat (white) | BLQ | BLQ | BLQ | 348 | BLQ | BLQ |
| Harderian gland | BLQ | 1983 | 1478 | BLQ | BLQ | BLQ |
| Kidney | 3154 | 3060 | 2704 | 2075 | 2031 | 1120 |
| Lacrimal gland (Ex-orbital) | BLQ | 1557 | 1655 | 1993 | 1388 | BLQ |
| Lacrimal gland (Intra-orbital) | BLQ | 2323 | 1355 | BLQ | BLQ | BLQ |
| Large intestinal contents | BLQ | 519 | NR | 810 | 593 | BLQ |
| Large intestine | BLQ | 1787 | 1716 | 1361 | BLQ | BLQ |
| Liver | 2431 | 3607 | 2005 | 1561 | 1190 | 715 |
| Lung | 920 | 1185 | 683 | 677 | 491 | 371 |
| Lymph node | 938 | 1376 | NR | BLQ | BLQ | BLQ |
| Medulla | 606 | 714 | 501 | BLQ | 323 | BLQ |
| Muscle | 563 | 732 | 773 | 638 | 401 | 341 |
| Myocardium | 1032 | 1908 | 1058 | 771 | 496 | 395 |
| Nasal turbinates | 941 | 1302 | 978 | 1055 | NR | 525 |
| Olfactory lobe | 708 | 747 | 466 | 725 | BLQ | BLQ |
| Pancreas | 1676 | 3492 | 2193 | 2404 | 641 | 385 |
| Preputial gland | 1660 | 1347 | 1073 | ND | ND | ND |
| Prostate gland | 675 | 1220 | 1045 | 995 | NR | 497 |
| Salivary gland | 1518 | 2826 | 2286 | 2458 | 1533 | 596 |
| Seminal vesicle | 326 | 469 | 591 | 513 | BLQ | BLQ |
| Skin | 863 | 857 | 531 | 717 | 445 | 464 |
| Small intestinal contents | 715 | 711 | 847 | 774 | 400 | BLQ |
| Small intestine | BLQ | BLQ | BLQ | BLQ | BLQ | BLQ |
| Spinal cord | 544 | 715 | BLQ | BLQ | 394 | BLQ |
| Spleen | 788 | 1058 | 810 | 796 | 736 | 556 |
| Stomach | BLQ | 1798 | ND | 935 | BLQ | BLQ |
| Stomach contents | BLQ | BLQ | BLQ | BLQ | BLQ | BLQ |
| Testes | 475 | 580 | 410 | 583 | BLQ | BLQ |
| Thymus | 801 | 1212 | 838 | NR | 654 | 596 |
| Thyroid gland | ND | 4809 | 3675 | NR | 644 | 589 |
| Urine | 3531 | 5000 | 3016 | 1974 | BLQ | BLQ |

BLQ: Below limit of quantitation (<320 ng equivalents test article/g).
ND: Not detectable (sample shape not discernible from background or surrounding tissue).
NR: Not represented (tissue not represented on section).

The concentrations of radioactivity in blood and tissues in rats treated with a single dose of oral ¹⁴C-citrate are shown in Table 11.

TABLE 11
Radioactivity in Tissues of Rats Treated with Ora ¹⁴C-Citrate

| | ng Equivalents test article/g | | | | | |
|---|---|---|---|---|---|---|
| | Animal Number (Timepoint) | | | | | |
| | 2001 | 2002 | 2003 | 2004 | 2005 | 2006 |
| Tissue | (0.5 Hour) | (1 Hour) | (2 Hours) | (4 Hours) | (8 Hours) | (24 Hours) |
| Adrenal gland | NR | 20538 | 17413 | 13375 | 15645 | 10316 |
| Blood | 15935 | 12808 | 11183 | 8800 | BLQ | BLQ |
| Bone | 49396 | 58936 | 43368 | 42499 | 47959 | 17943 |
| Bone marrow | 9150 | 18940 | 21841 | 20023 | 21000 | 11005 |
| Cecum | BLQ | ND | ND | 21290 | ND | BLQ |
| Cecum contents | 3593 | 7501 | 9083 | 37320 | 8242 | 4081 |
| Cerebellum | 5447 | 5944 | 6952 | 5953 | 4300 | 3183 |
| Cerebrum | 4464 | 5907 | 6789 | 5659 | 4073 | 3231 |
| Epididymis | 4469 | 6854 | 6691 | 7917 | 6583 | 4188 |
| Esophageal contents | 3395667 | NR | 455859 | NR | BLQ | BLQ |
| Eye | BLQ | 3086 | 4533 | 6017 | BLQ | BLQ |
| Fat (brown) | BLQ | BLQ | BLQ | BLQ | BLQ | BLQ |
| Fat (white) | BLQ | BLQ | BLQ | BLQ | BLQ | BLQ |
| Harderian gland | 7011 | NR | 17809 | 22082 | 30362 | 11021 |
| Kidney | 31411 | 35017 | 25508 | 21332 | 17379 | 8211 |
| Lacrimal gland (Ex-orbital) | 9751 | 13243 | 16362 | 21167 | 17875 | 7542 |
| Lacrimal gland (Intra-orbital) | NR | 27199 | 23343 | 20093 | 22551 | NR |
| Large intestinal contents | 3638 | 4169 | 5302 | 11545 | 12377 | 5971 |
| Large intestine | BLQ | 16238 | 15856 | 28662 | 11353 | BLQ |
| Liver | 35567 | 34866 | 31094 | 22356 | 20156 | 10050 |
| Lung | 11418 | 11240 | 10832 | 8280 | 6727 | 4911 |
| Lymph node | BLQ | BLQ | BLQ | BLQ | BLQ | BLQ |
| Medulla | 4578 | 6118 | 7787 | 6270 | 4556 | 3479 |
| Muscle | 3783 | 5745 | 4737 | 4311 | 3965 | 3490 |
| Myocardium | 7466 | 8932 | 9941 | 7845 | 5336 | 4390 |
| Nasal turbinates | 12703 | 12918 | 14959 | 24265 | 9529 | NR |
| Olfactory lobe | NR | 5917 | 8491 | 8727 | NR | 4121 |
| Pancreas | 22855 | 49518 | 48038 | 52562 | 13125 | 5990 |
| Preputial gland | ND | ND | ND | ND | ND | ND |
| Prostate gland | 4912 | 8819 | 8011 | 12828 | 7804 | 7448 |
| Salivary gland | 12638 | 24116 | 28408 | 35370 | 33304 | 6456 |
| Seminal vesicle | 3710 | 4087 | 5590 | NR | 8840 | NR |
| Skin | 7114 | 10352 | 9019 | 8095 | 7843 | 6049 |
| Small intestinal contents | 765641 | 155596 | 79444 | 61651 | 25276 | 4687 |
| Small intestine | ND | 11950 | ND | ND | ND | 21231 |
| Spinal cord | 3633 | 4913 | 6114 | 6416 | 5079 | 3837 |
| Spleen | 8138 | 11559 | 12833 | 11528 | 14371 | 7745 |
| Stomach | 120191 | 57312 | 20761 | 14630 | ND | 6169 |
| Stomach contents | 3239644 | 1081479 | 856445 | 88064 | BLQ | BLQ |
| Testes | 3146 | 5217 | 5382 | 5009 | 4224 | 3751 |
| Thymus | 5369 | 9433 | 10568 | 10357 | 10658 | 8768 |
| Thyroid gland | 6773 | 22240 | 9678 | 5807 | NR | BLQ |
| Urine | 54074 | 145954 | NR | 119053 | NR | 3684 |

BLQ: Below limit of quantitation (<320 ng equivalents test article/g).
ND: Not detectable (sample shape not discernible from background or surrounding tissue).
NR: Not represented (tissue not represented on section).

### Example 4: Oral Bioavailability of Succinate

Summary

The oral bioavailability of ¹³C₄-succinate was evaluated in male Sprague-Dawley rats. ¹³C₄-succinate was dosed by intravenous (IV) and oral (PO) routes of administration at 10 mg/kg and 50 mg/kg, respectively. Blood samples were collected up to 8 hours post-dose, and plasma concentrations of the test articles were determined by LC-MS/MS. Pharmacokinetic parameters were determined using Phoenix WinNonlin (v8.0) software.

Following IV dosing of ¹³C₄-succinate at 10 mg/kg, the average half-life was 2.46±2.04 hours. Its average clearance rate was 4.61±0.341 L/hr/kg. The average volume of distribution was 0.982±0.584 L/kg. Following PO dosing of ¹³C₄-succinate at 50 mg/kg, maximum plasma concentrations (average of 211±186 ng/mL) were observed at 15 minutes post dosing. The average half-life following oral dosing could not be determined because the correlation coefficient (r²) was less than 0.85, or due to a lack of quantifiable data points trailing the Cₘₐₓ. The average exposure based on the dose-normalized AUCₗₐₛₜ was 1.84±1.58 hr*kg*ng/mL/mg. The average oral bioavailability of ¹³C₄-succinate after dosing at 50 mg/kg was 0.854±0.735%.

Observations and Adverse Reactions

No adverse reactions were observed following intravenous and oral dosing of ¹³C₄-succinate in male Sprague-Dawley rats.

Dosing Solution Analysis

The dosing solutions were analyzed by LC-MS/MS. The measured dosing solution concentrations are shown in Table 12. The dosing solutions were diluted into rat plasma and analyzed in triplicate. All concentrations are expressed as mg/mL of the free base. The nominal dosing level was used in all calculations.

TABLE 12
Measured Dosing Solution Concentrations (mg/mL)

| | | | Nominal | Measured | |
|---|---|---|---|---|---|
| | Route of | | Dosing | Dosing | |
| Test | Adminis- | | Conc. | Conc. | % of |
| Article | tration | Vehicle | (mg/mL) | (mg/mL) | Nominal |
| ¹³C₄- | IV | D5W* | 2 | 2.06 | 103 |
| succinate | PO | Water | 10 | 9.7 | 97.0 |

*5% dextrose solution

Quantitative Plasma Sample Analysis

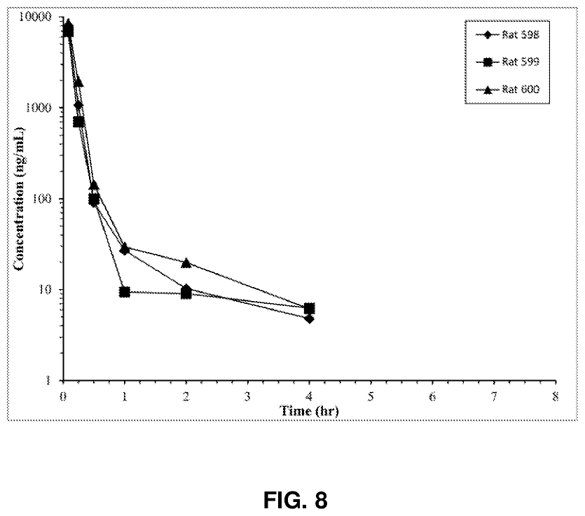

Plasma samples were extracted and analyzed using the methods described in the section on Sample Extraction. Individual and average plasma concentrations are shown in Table 13 and Table 14. All data are expressed as ng/mL of the free base. Samples that were below the limit of quantification were not used in the calculation of averages. Concentrations versus time data are plotted in FIGS. 8-11.

Data Analysis

Pharmacokinetic parameters were calculated from the time course of the plasma concentration and are presented in Table 13 and Table 14. Pharmacokinetic parameters were determined with Phoenix WinNonlin (v8.0) software using a non-compartmental model. The maximum plasma concentrations (C₀) after IV dosing were estimated by extrapolation of the first two time points back to t=0. The maximum plasma concentration (Cₘₐₓ) and the time to reach maximum plasma concentration (Tₘₐₓ) after PO dosing were observed from the data. The area under the time concentration curve (AUC) was calculated using the linear trapezoidal rule with calculation to the last quantifiable data point, and with extrapolation to infinity if applicable. Plasma half-life (t_{1/2}) was calculated from 0.693/slope of the terminal elimination phase. Mean residence time, MRT, was calculated by dividing the area under the moment curve (AUMC) by the AUC. Clearance (CL) was calculated from dose/AUC. Steady-state volume of distribution (Vss) was calculated from CL*MRT (mean residence time). Bioavailability was determined by dividing the individual dose-normalized PO AUCₗₐₛₜ values by the average dose-normalized IV AUCₗₐₛₜ value. Any samples below the limit of quantitation (1 ng/mL) were treated as zero for pharmacokinetic data analysis.

TABLE 13
Individual and Average Plasma Concentrations (ng/mL) and
Pharmacokinetic Parameters for ¹³C₄-succinate After Intravenous
Administration at 10 mg/kg in Male Sprague-Dawley Rats
Intravenous (10 mg/kg)

| | Rat # | | | | |
|---|---|---|---|---|---|
| Time (hr) | Rat 598 | Rat 599 | Rat 600 | Mean | SD |
| 0 (pre-dose) | BLOQ | BLOQ | BLOQ | ND | ND |
| 0.083 | 8050 | 6880 | 8410 | 7780 | 800 |
| 0.25 | 1070 | 702 | 1940 | 1237 | 636 |
| 0.50 | 91.0 | 99.8 | 144 | 112 | 28.4 |
| 1.0 | 27.0 | 9.43 | 29.5 | 22.0 | 10.,9 |
| 2.0 | 10.3 | 9.06 | 19.9 | 13.1 | 5.93 |
| 4.0 | 4.79 | 6.25 | 6.15 | 5.73 | 0.816 |
| 8.0 | BLOQ | BLOQ | BLOQ | ND | ND |
| Animal Weight (kg) | 0.294 | 0.298 | 0.310 | 0.301 | 0.008 |
| Volume Dosed (mL) | 1.47 | 1.49 | 1.55 | 1.50 | 0.04 |
| C₀ (ng/mL)¹ | 21947 | 21392 | 17434 | 20257 | 2461 |
| tₘₐₓ (hr)¹ | 0 | 0 | 0 | 0 | 0 |
| t_{1/2} (hr) | 1.26 | 4.81 | 1.30 | 2.46 | 2.04 |
| MRTₗₐₛₜ (hr) | 0.102 | 0.0949 | 0.140 | 0.113 | 0.0244 |
| CL (L/hr/kg) | 4.50 | 5.00 | 4.34 | 4.61 | 0.341 |
| Vₛₛ (L/kg) | 0.561 | 1.65 | 0.735 | 0.982 | 0.584 |
| AUCₗₐₛₜ (hr · ng/mL) | 2215 | 1958 | 2291 | 2155 | 174 |
| AUC_{∞} (hr · ng/mL) | 2223 | 2902 | 2303 | 2176 | 156 |
| Dose-normalized Values² | | | | | |
| AUCₗₐₛₜ (hr · kg · ng/mL/mg) | 221 | 196 | 229 | 215 | 17.4 |
| AUC_{∞} (hr · kg · ng/mL/mg) | 222 | 290 | 230 | 218 | 15.6 |

C₀: maximum plasma concentration extrapolated to t = 0; tₘₐₓ: time of maximum plasma concentration; t_{1/2}: half-life, data points used for half-life determination are in bold; MRTₗₐₛₜ: mean residence time, calculated to the last observable time point; CL: clearance; Vₛₛ: steady state volume of distribution; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Extrapolated to t = 0.
²Dose-normalized by dividing the parameter by the nominal dose in mg/kg.

FIG. 8 is a graph of plasma concentration of ¹³C₄-succinate at various time points in individual rats after intravenous administration of ¹³C₄-succinate at 10 mg/kg.

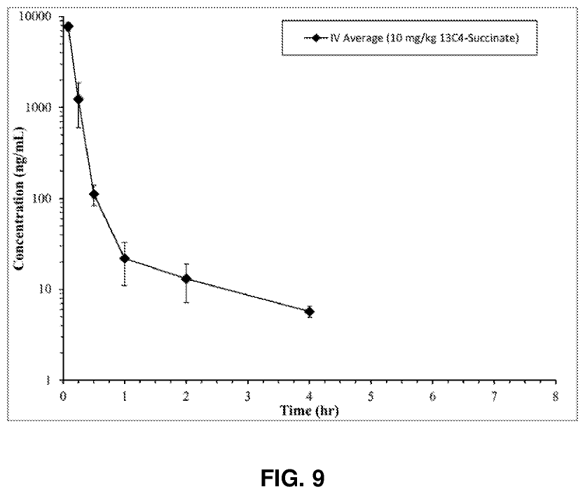

FIG. 9 is a graph of average plasma concentration of ¹³C₄-succinate at various time points in rats after intravenous administration of ¹³C₄-succinate at 10 mg/kg.

TABLE 14
Individual and Average Plasma Concentrations (ng/mL) for ¹³C₄-succinate
After Oral Administration at 50 mg/kg in Male Sprague-Dawley Rats
Oral (50 mg/kg)

| | Rat # | | | | |
|---|---|---|---|---|---|
| Time (hr) | Rat 601 | Rat 602 | Rat 603 | Mean | SD |
| 0 (pre-dose) | BLOQ | BLOQ | BLOQ | ND | ND |
| 0.25 | 186 | 37.5 | 408 | 211 | 186 |
| 0.50 | 75.4 | 27.8 | 186 | 96.4 | 81.2 |
| 1.0 | 3.78 | 3.00 | 5.66 | 4.15 | 1.37 |
| 2.0 | 1.29 | BLOQ | 2.25 | 1.77 | ND |
| 4.0 | BLOQ | BLOQ | BLOQ | ND | ND |
| 8.0 | BLOQ | BLOQ | BLOQ | ND | ND |
| Animal Weight (kg) | 0.285 | 0.311 | 0.311 | 0.302 | 0.015 |
| Volume Dosed (mL) | 1.43 | 1.56 | 1.56 | 1.52 | 0.08 |
| Cₘₐₓ (ng/mL) | 186 | 37.5 | 408 | 211 | 186 |
| tₘₐₓ (hr) | 0.25 | 0.25 | 0.25 | 0.25 | 0.00 |
| t_{1/2} (hr) | ND³ | ND⁴ | ND³ | ND | ND |
| MRTₗₐₛₜ (hr) | 0.382 | 0.404 | 0.378 | 0.388 | 0.0143 |
| AUCₗₐₛₜ (hr · ng/mL) | 78.3 | 20.6 | 177 | 92.0 | 79.2 |
| AUC_{∞} (hr · ng/mL) | ND³ | ND⁴ | ND³ | ND | ND |
| Dose-normalized | | | | | |
| Values¹ | | | | | |
| AUCₗₐₛₜ | 1.57 | 0.411 | 3.54 | 1.84 | 1.58 |
| (hr · kg · ng/mL/mg) | | | | | |
| AUC_{∞} | ND³ | ND⁴ | ND³ | ND | ND |
| (hr · kg · ng/mL/mg) | | | | | |
| Bioavailability (%)² | 0.726 | 0.191 | 1.64 | 0.854 | 0.735 |

Cₘₐₓ: maximum plasma concentration; tₘₐₓ: time of maximum plasma concentration; t_{1/2}: half-life, data points used for half-life determination are in bold; MRTₗₐₛₜ: mean residence time, calculated to the last observable time point; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Dose-normalized by dividing the parameter by the nominal dose in mg/kg.
²Bioavailability determined by dividing the individual oral AUCₗₐₛₜ values by the average IV AUCₗₐₛₜ value.
³Not determined because the line defining the terminal elimination phase had an r² < 0.85.
⁴Not determined due to a lack of quantifiable data points trailing the Cₘₐₓ.

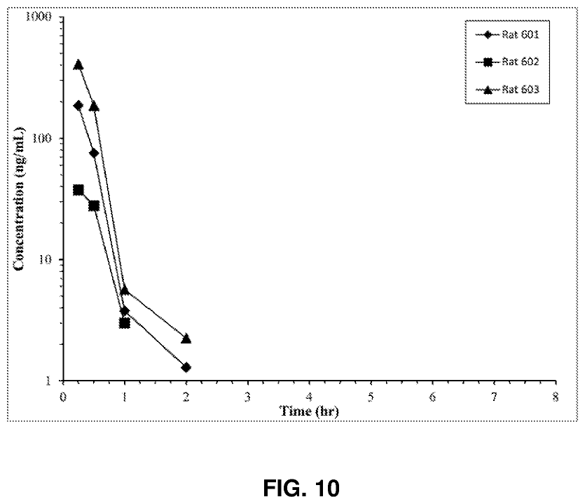

FIG. 10 is a graph of plasma concentration of ¹³C₄-succinate at various time points in individual rats after oral administration of ¹³C₄-succinate at 50 mg/kg.

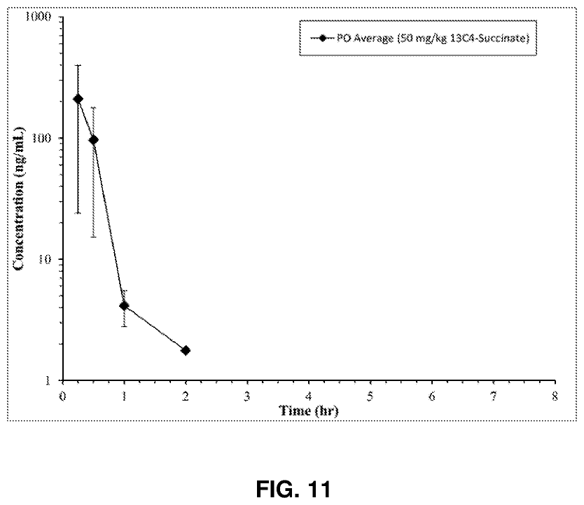

FIG. 11 is a graph of average plasma concentration of ¹³C₄-succinate at various time points in rats after oral administration of ¹³C₄-succinate at 50 mg/kg.

Analytical Stock Solution Preparation

Analytical stock solutions (1.00 mg/mL of the free drug) were prepared in DMSO.

Standard Preparation

Standards were prepared in Sprague-Dawley rat plasma containing sodium heparin as the anticoagulant. Working solutions were prepared in 50:50 acetonitrile:water and then added to the rat plasma to make calibration standards to final concentrations of 2000, 1000, 500, 100, 50.0, 10.0, 5.00, 2.50, and 1.00 ng/mL. Standards were treated identically to the study samples.

Sample Extraction

Plasma samples were manually extracted using acetonitrile in a 96-well plate as outlined in Table 15.

TABLE 15
Preparation of Plasma Samples

| Step | Procedure |
|---|---|
| 1 | Standards: Add 10 μL of appropriate working solution to 50 μL |
| | of blank plasma. |
| | Blanks: Add 10 μL 50:50 acetonitrile:water to 50 μL of blank |
| | plasma. |
| | Samples: Add 10 μL 50:50 acetonitrile:water to 50 μL of study |
| | sample. |
| | Cap and mix. |
| 2 | Add 150 μL of acetonitrile containing 100 ng/mL of warfarin as |
| | internal standard. |
| | Cap and vortex well. |
| 3 | Centrifuge samples at 3000 rpm for 5 minutes. |
| 4 | Evaporate 100 μL of supernatant and reconstitute with 100 μL |
| | of Milli-Q water. |

### Example 5: Oral Bioavailability of Citrate

Summary

The oral bioavailability of ¹³C₆-citrate was evaluated in male Sprague-Dawley rats. ¹³C₆-citrate was dosed by intravenous (IV) and oral (PO) routes of administration at 10 mg/kg and 50 mg/kg, respectively. Blood samples were collected up to 8 hours post-dose, and plasma concentrations of the test articles were determined by LC-MS/MS. Pharmacokinetic parameters were determined using Phoenix WinNonlin (v8.0) software.

Following IV dosing of ¹³C₆-citrate at 10 mg/kg, the average half-life was 2.34±0.207 hours. Its average clearance rate was 2.96±0.345 L/hr/kg. The average volume of distribution was 1.75±0.223 L/kg. Following PO dosing of ¹³C₆-citrate at 50 mg/kg, maximum plasma concentrations (average of 1580±191 ng/mL) were observed at 15 minutes post dosing. The average half-life following oral dosing could not be determined; however, the half-life for one rat was 0.865 hours. The average exposure based on the dose-normalized AUCₗₐₛₜ was 33.2±10.9 hr*kg*ng/mL/mg. The average oral bioavailability of ¹³C₆-citrate after dosing at 50 mg/kg was 9.86±3.24%.

Observations and Adverse Reactions

No adverse reactions were observed following intravenous and oral dosing of ¹³C₆-citrate in male Sprague-Dawley rats.

Dosing Solution Analysis

The dosing solutions were analyzed by LC-MS/MS. The measured dosing solution concentrations are shown in Table 16. The dosing solutions were diluted into rat plasma and analyzed in triplicate. All concentrations are expressed as mg/mL of the free base. The nominal dosing level was used in all calculations.

TABLE 16
Measured Dosing Solution Concentrations (mg/mL)

| | | | Nominal | Measured | |
|---|---|---|---|---|---|
| | Route of | | Dosing | Dosing | |
| Test | Adminis- | | Conc. | Conc. | % of |
| Article | tration | Vehicle | (mg/mL) | (mg/mL) | Nominal |
| ¹³C₆- | IV | D5W* | 2 | 2.40 | 120 |
| Citrate | PO | Water | 10 | 8.73 | 87.3 |

*5% dextrose solution

Quantitative Plasma Sample Analysis

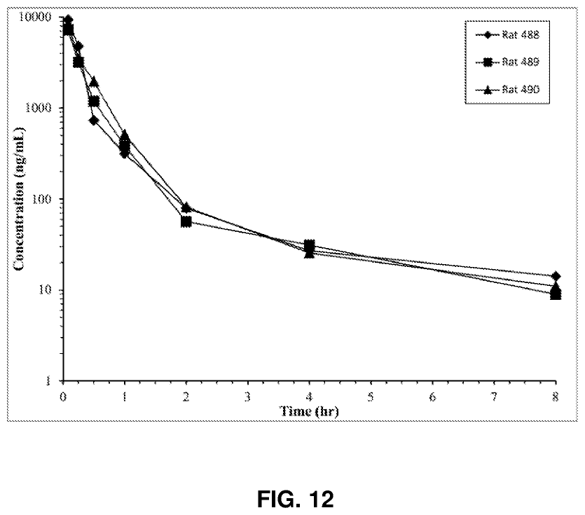

Plasma samples were extracted and analyzed using the methods described in the section on Sample Extraction. Individual and average plasma concentrations are shown in Table 17 and Table 18. All data are expressed as ng/mL of the free base. Samples that were below the limit of quantification were not used in the calculation of averages. Concentrations versus time data are plotted in FIGS. 12-15.

Data Analysis

Pharmacokinetic parameters were calculated from the time course of the plasma concentration and are presented in Table 17 and Table 18. Pharmacokinetic parameters were determined with Phoenix WinNonlin (v8.0) software using a non-compartmental model. The maximum plasma concentrations (C₀) after IV dosing were estimated by extrapolation of the first two time points back to t=0. The maximum plasma concentration (Cₘₐₓ) and the time to reach maximum plasma concentration (Tₘₐₓ) after PO dosing were observed from the data. The area under the time-concentration curve (AUC) was calculated using the linear trapezoidal rule with calculation to the last quantifiable data point, and with extrapolation to infinity if applicable. Plasma half-life (t_{1/2}) was calculated from 0.693/slope of the terminal elimination phase. Mean residence time, MRT, was calculated by dividing the area under the moment curve (AUMC) by the AUC. Clearance (CL) was calculated from dose/AUC. Steady-state volume of distribution (Vss) was calculated from CL*MRT (mean residence time). Bioavailability was determined by dividing the individual dose-normalized PO AUCₗₐₛₜ values by the average dose-normalized IV AUCₗₐₛₜ value. Any samples below the limit of quantitation (1 ng/mL) were treated as zero for pharmacokinetic data analysis.

TABLE 17
Individual and Average Plasma Concentrations (ng/mL) and
Pharmacokinetic Parameters for ¹³C₆-citrate After Intravenous
Administration at 10 mg/kg in Male Sprague-Dawley Rats
Intravenous (10 mg/kg)

| Time | Rat # | | | | |
|---|---|---|---|---|---|
| (hr) | Rat 488 | Rat 480 | Rat 490 | Mean | SD |
| 0 (pre-dose) | BLOQ | BLOQ | BLOQ | ND | ND |
| 0.083 | 9380 | 7270 | 8540 | 8397 | 1062 |
| 0.25 | 4780 | 3180 | 3550 | 3837 | 838 |
| 0.50 | 733 | 1190 | 1960 | 1294 | 620 |
| 1.0 | 316 | 388 | 511 | 405 | 98.6 |
| 2.0 | 79.5 | 56.6 | 81.7 | 72.6 | 13.9 |
| 4.0 | 27.0 | 31.2 | 25.5 | 27.9 | 2.95 |
| 8.0 | 14.2 | 8.99 | 11.0 | 11.4 | 2.63 |
| Animal Weight (kg) | 0.290 | 0.293 | 0.292 | 0.292 | 0.002 |
| Volume Dosed (mL) | 1.45 | 1.47 | 1.46 | 1.46 | 0.01 |
| C₀ (ng/mL)¹ | 13113 | 10965 | 13211 | 12430 | 1269 |
| tₘₐₓ (hr)¹ | 0 | 0 | 0 | 0 | 0 |
| t_{1/2} (hr) | 2.58 | 2.26 | 2.19 | 2.34 | 0.207 |
| MRTₗₐₛₜ (hr) | 0.437 | 0.488 | 0.474 | 0.466 | 0.0259 |
| CL (L/hr/kg) | 2.85 | 3.34 | 2.68 | 2.96 | 0.345 |
| Vₛₛ (L/kg) | 1.73 | 1.98 | 1.54 | 1.75 | 0.223 |
| AUCₗₐₛₜ (hr · ng/mL) | 3454 | 2961 | 3695 | 3370 | 374 |
| AUC_{∞} (hr · ng/mL) | 3507 | 2999 | 3730 | 3409 | 380 |
| Dose-normalized Values² | | | | | |
| AUCₗₐₛₜ (hr · kg · ng/mL/mg) | 345 | 296 | 370 | 337 | 37.4 |
| AUC_{∞} (hr · kg · ng/mL/mg) | 351 | 299 | 373 | 341 | 38.0 |

C₀: maximum plasma concentration extrapolated to t = 0; tₘₐₓ: time of maximum plasma concentration; t_{1/2}: half-life, data points used for half-life determination are in bold; MRTₗₐₛₜ: mean residence time, calculated to the last observable time point; CL: clearance; Vₛₛ: steady state volume of distribution; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Extrapolated to t = 0.
²Dose-normalized by dividing the parameter by the nominal dose in mg/kg.

FIG. 12 is a graph of plasma concentration of ¹³C₆-citrate at various time points in individual rats after intravenous administration of ¹³C₆-citrate at 10 mg/kg.

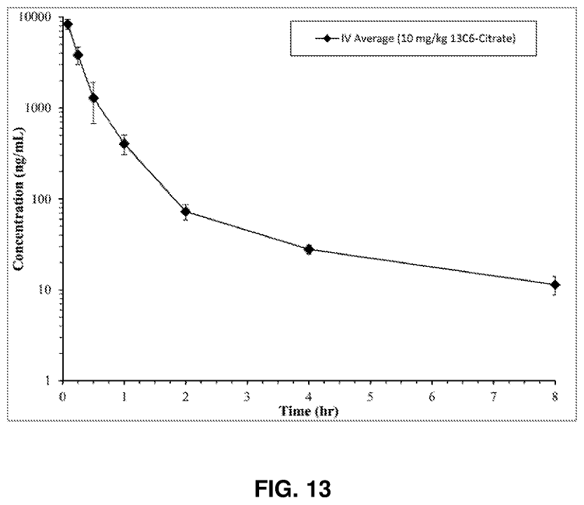

FIG. 13 is a graph of average plasma concentration of ¹³C₆-citrate at various time points in rats after intravenous administration of ¹³C₆-citrate at 10 mg/kg.

TABLE 18
Individual and Average Plasma Concentrations (ng/mL) for ¹³C₆-citrate After
Oral Administration at 50 mg/kg in Male Sprague-Dawley Rats
Oral (50 mg/kg)

| | Rat # | | | | |
|---|---|---|---|---|---|
| Time (hr) | Rat 491 | Rat 492 | Rat 493 | Mean | SD |
| 0 (pre-dose) | BLOQ | BLOQ | BLOQ | ND | ND |
| 0.25 | 1800 | 1470 | 1470 | 1580 | 191 |
| 0.50 | 1770 | 1440 | 909 | 1373 | 434 |
| 1.0 | 1090 | 740 | 420 | 750 | 335 |
| 2.0 | 147 | 98.6 | 44.4 | 96.7 | 51.3 |
| 4.0 | 16.5 | 11.6 | 7.06 | 11.7 | 4.72 |
| 8.0 | 4.43 | 4.72 | 4.14 | 4.43 | 0.290 |
| Animal Weight (kg) | 0.286 | 0.281 | 0.278 | 0.282 | 0.004 |
| Volume Dosed (mL) | 1.43 | 1.41 | 1.39 | 1.41 | 0.02 |
| Cₘₐₓ (ng/mL) | 1800 | 1470 | 1470 | 1580 | 191 |
| tₘₐₓ (hr) | 0.25 | 0.25 | 0.25 | 0.25 | 0.00 |
| t_{1/2} (hr) | 0.865 | ND³ | ND³ | ND | ND |
| MRTₗₐₛₜ (hr) | 0.892 | 0.863 | 0.770 | 0.841 | 0.0640 |
| AUCₗₐₛₜ (hr · ng/mL) | 2210 | 1655 | 1119 | 1661 | 545 |
| AUC_{∞} (hr · ng/mL) | 2216 | ND³ | ND³ | ND | ND |
| Dose-normalized Values¹ | | | | | |
| AUCₗₐₛₜ (hr · kg · ng/mL/mg) | 44.2 | 33.1 | 22.4 | 33.2 | 10.9 |
| AUC_{∞} (hr · kg · ng /mL/mg) | 44.3 | ND³ | ND³ | ND | ND |
| Bioavailability (%)² | 13.1 | 9.82 | 6.64 | 9.86 | 3.24 |

Cₘₐₓ: maximum plasma concentration; tₘₐₓ: time of maximum plasma concentration; t_{1/2}: half-life, data points used for half-life determination are in bold; MRTₗₐₛₜ: mean residence time, calculated to the last observable time point; AUCₗₐₛₜ: area under the curve, calculated to the last observable time point; AUC_{∞}: area under the curve, extrapolated to infinity; ND: not determined; BLOQ: below the limit of quantitation (1 ng/mL).
¹Dose-normalized by dividing the parameter by the nominal dose in mg/kg.
²Bioavailability determined by dividing the individual oral AUCₗₐₛₜ values by the average IV AUCₗₐₛₜ value.
³Not determined because the line defining the terminal elimination phase had an r² < 0.85.

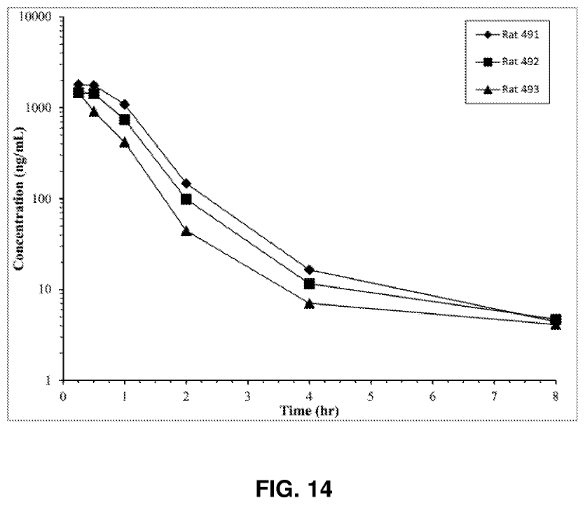

FIG. 14 is a graph of plasma concentration of ¹³C₆-Citrate at various time points in individual rats after oral administration of ¹³C₆-Citrate at 50 mg/kg.

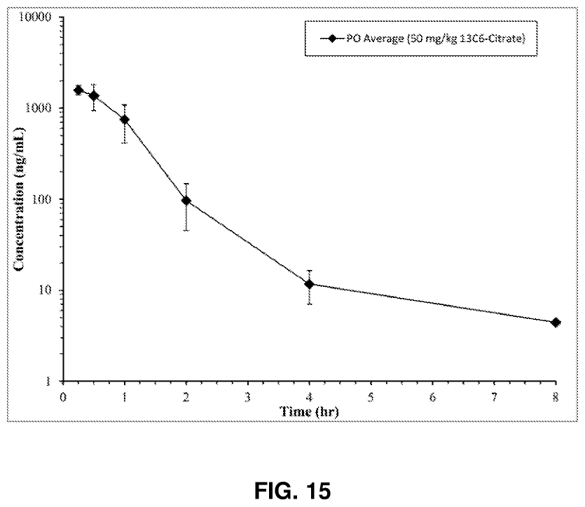

FIG. 15 is a graph of average plasma concentration of ¹³C₄-succinate at various time points in rats after oral administration of ¹³C₆-Citrate at 50 mg/kg.

Analytical Stock Solution Preparation

Analytical stock solutions (1.00 mg/mL of the free drug) were prepared in DMSO.

Standard Preparation

Standards were prepared in Sprague-Dawley rat plasma containing sodium heparin as the anticoagulant. Working solutions were prepared in 50:50 acetonitrile:water and then added to the rat plasma to make calibration standards to final concentrations of 2000, 1000, 500, 100, 50.0, 10.0, 5.00, 2.50, and 1.00 ng/mL. Standards were treated identically to the study samples.

Sample Extraction

Plasma samples were manually extracted using acetonitrile in a 96-well plate as outlined in Table 19.

TABLE 19
Preparation of Plasma Samples

| Step | Procedure |
|---|---|
| 1 | Standards: Add 10 μL of appropriate working solution to 50 μL |
| | of blank plasma. |
| | Blanks: Add 10 μL 50:50 acetonitrile:water to 50 μL of blank |
| | plasma. |
| | Samples: Add 10 μL 50:50 acetonitrile:water to 50 μL of study |
| | sample. |
| | Cap and mix. |
| 2 | Add 150 μL of acetonitrile containing 100 ng/mL of warfarin as |
| | internal standard. |
| | Cap and vortex well. |
| 3 | Centrifuge samples at 3000 rpm for 5 minutes. |
| 4 | Evaporate 100 μL of supernatant and reconstitute with 100 μL |
| | of Milli-Q water. |

### Example 6: Solubility of Citric Acid in Lysine-Buffered Solutions

The effect of lysine buffers on the solubility of citric acid at various pH values was analyzed. Results are shown in Table 20.

TABLE 20
Solubility of Citric Acid in Lysine-buffered Solutions

| | | | | | Final citric |
|---|---|---|---|---|---|
| Molar ratio of | Citric | | | | acid |
| citric | acid | L-lysine | Water | Final | concentration |
| acid:lysine | (mg) | (mg) | (mL) | pH | (mg/mL) |
| 1:1 | 213 | 163 | 0.5 | 3.5 | 426 |
| 1:2 | 214 | 318 | 0.5 | 4.8 | 428 |
| 1:2.5 | 256 | 492 | 0.6 | 5.6 | 427 |
| 1:3 | 212 | 474 | 0.8 | 6.4 | 265 |
| 1:4 | 216 | 639 | 1 | 9.4 | 216 |

### Example 7: Citrate-Containing Formulations

Citrate-containing containing formulations according to embodiments of the invention are provided in Table 21.

TABLE 21
Citrate-containing Formulations

Mass (g)

| Component | Formula 1 | Formula 2 |
|---|---|---|
| monosodium citrate | 1.35 | 5.4 |
| monopotassium citrate | 0.22 | 1.475 |
| citric acid | 33.61 | 28.91 |
| L-lysine, free base | 25.69 | 22.1 |
| sucrose | 7.5 | 7.5 |

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1-124. (canceled)

125. A method of treating a condition associated with altered TCA cycle metabolism in a subject, the method comprising providing to a subject having a condition associated with altered TCA cycle metabolism a composition comprising a prodrug, analog, or derivative of succinate.

126. The method of claim 125, wherein the composition is formulated for non-oral administration.

127. The method of claim 126, wherein the composition is provided to the subject subcutaneously, intravenously, intraarterially, intramuscularly, intradermally, or rectally.

128. The method of claim 126, wherein the composition further comprises a buffering agent in an amount to buffer a pH of the composition from about 3.0 to about 8.0.

129. The method of claim 128, wherein the buffering agent comprises an amino acid.

130. The method of claim 128, wherein the buffering agent comprises a metal ion.

131. The method of claim 125, wherein the composition is formulated for oral administration.

132. The method of claim 125, wherein the prodrug, analog, or derivative of succinate is provided at from about 1 mg/kg subject weight to about 5 g/kg subject weight.

133. The method of claim 125, wherein the condition is selected from the group consisting of a disorder related to POLG mutation, an energetic disorder, glutaric acidemia type 1 or type 2, a long chain fatty acid oxidation disorder, methylmalonic acidemia (MMA), a mitochondrial associated disease, a mitochondrial encephalomyopathy lactic acidosis and stroke-like syndrome (MELAS), myoclonic epilepsy and ragged-red fibers (MERRF), mitochondrial myopathy, a mitochondrial respiratory chain deficiency, muscular dystrophy (e.g., Duchenne's muscular dystrophy and Becker's muscular dystrophy), a neurologic disorder, a pain or fatigue disease, propionic acidemia (PA), pyruvate carboxylase deficiency, refractory epilepsy, and succinyl CoA lyase deficiency.

134. The method of claim 125, wherein the composition is provided in multiple doses per day.
